SEQUENCE LISTING (69 sequences)

SEQ ID NO: 1 - DNA, 100 bp, Homo sapiens
atcgcactcatgtggaagttcggcggcatctacctggacacggacttcattgttctcaagaacctgcggaacctgaccaacgtgctgggcacccagtccc

SEQ ID NO: 2 - DNA, 100 bp, Homo sapiens
agagctgggctttcccggggtccaaattggcacccacgtcaacgagtgggacctgaacgcgcaggagctctttcctgtctatgcggcagccgaaaggctg

SEQ ID NO: 3 - DNA, 100 bp, Homo sapiens
cctggggccgggtacgaggatgtcgtctggattcccaaaggctccgtccacatcttcatccaggatctgaacctctctctcagtcacttggccctgaagg

SEQ ID NO: 4 - DNA, 100 bp, Homo sapiens
cctgtggaaatttgtggaccctgggcaccctctcttgctcccaaattttaatcggctcctggaaacctcaccccaaattggagataggcactcctcttgt

SEQ ID NO: 5 - DNA, 100 bp, Homo sapiens
gaaaggaaggagaggagaaaactgcactggatcaaggaggtcttcccataggcctgcagtagggctgtttaccattggaagcaagggaaagaggaggaga

SEQ ID NO: 6 - DNA, 100 bp, Homo sapiens
aatgcctttaaaccaaatatcctactgatcatggcggatgatctaggcactggggatctcggttgctacgggaacaatacactgagaacgccgaatattg

SEQ ID NO: 7 - DNA, 100 bp, Homo sapiens
tatggaagcccactgtggacaaactgagcttactgaaagtctgaagaccaaagcttttcaggctcacactccagcacagaaagcttcagtcctggctttc

SEQ ID NO: 8 - DNA, 100 bp, Homo sapiens
aatggacctggagagtcaaatagaaagtctgaaagaagaacttggctctctatcaagaaactatgaagaggatgtgaagctgctgcacaaacagttggca

SEQ ID NO: 9 - DNA, 100 bp, Homo sapiens
atctgcctcttctacctagcaaagtagatctccagcaggtcaccataattccacacgatgagtggaaaaggattcaagatagccttgacaggttgacaag

SEQ ID NO: 10 - DNA, 100 bp, Homo sapiens
agagatgggggcgggaagagattcagctcccatccctccttcctctccttctccaagtgccttcaaaccaagaactgtacattcttctggttcctcagtg

SEQ ID NO: 11 - DNA, 100 bp, Homo sapiens
tcatattgagaggaatatgggcttgatcctcttcctatctaaatgggtgggccatttgattgtagagggtccaccacagaattatgggatgccttaagtg

SEQ ID NO: 12 - DNA, 100 bp, Homo sapiens
tctgctagaacagtgccgtgcttttccacagaaggttagaccctgaaagagatggctcagcaccacctatggatcttgctcctttgcctgcaaacctggc

SEQ ID NO: 13 - DNA, 100 bp, Homo sapiens
gctcagggctctttcctccacaccattcaggtctttctttccgaggcccctgtctcagggtgaggtgcttgagtctccaacggcaagggaacaagtactt

SEQ ID NO: 14 - DNA, 100 bp, Homo sapiens
aagcgcacattcatgtgggcatttcttgcgagcctcgcagcctccggaagctgtcgacttcatgacaagcattttgtgaactagggaagctcaggggggt

SEQ ID NO: 15 - DNA, 100 bp, Homo sapiens
tgggctgcgtggcgggcgacgaggagtcctacgaagtgttcaaggatctcttcgaccccatcatcgaggaccggcacggcggctacaagcccagcgatga

SEQ ID NO: 16 - DNA, 100 bp, Homo sapiens
ctggaagtgagggagtcaagggcaacaggggtttccctgggttaatgggtgaagatggcattaagggacagaaaggggacattggccctccaggatttcg

SEQ ID NO: 17 - DNA, 100 bp, Homo sapiens
tatatgggagtggaaagaaatacattggtccttgtggaggaagagattgctctgtttgccactgtgttcctgaaaaggggtctcggggtccaccaggacc

SEQ ID NO: 18 - DNA, 100 bp, Homo sapiens
atatgatcgcttgctggagacctgctctattgcccttgtgggcaaatacacgaagttctcagactcctatgcctctgtcattaaggctctggagcattct

SEQ ID NO: 19 - DNA, 100 bp, Homo sapiens
aacttctcaaaggccgaggagtgaccttcctatttcctatacaagcaaagacattccatcatgtttacagcgggaaggacttaattgcacaggcacggac

SEQ ID NO: 20 - DNA, 100 bp, Homo sapiens
cccgtgtcaacttctttctccctggcggtgaccacctggttctgctaaatgtttacacacagtgggctgagagtggttactcttcccagtggtgctatga

SEQ ID NO: 21 - DNA, 100 bp, Homo sapiens
tcgttttcttaagctgctgggtgctttgctgctcctggctctggccctctttttgggctttctacagttgggatggcggtttctggtgggactaggtgac

SEQ ID NO: 22 - DNA, 100 bp, Homo sapiens
gaggcataccaagatccacttgcggcagaaggacaagaaagcagacaaaagtgttgtggcctcttcggccacctcctctctctcttcctacccgtccccg

SEQ ID NO: 23 - DNA, 100 bp, Homo sapiens
tatatttggacacaagccttctccaggtgtggttcccaacaaaggtcagtttcccttggctgtgggagcccaggagttgtttctgtgcactggtcctatg

SEQ ID NO: 24 - DNA, 100 bp, Homo sapiens
cttttatcttctagccacagttctaagacccaaagtggggggtggacacagaagtccctaggagggaaggagctgttggggcaggggtgtaaattatttc

SEQ ID NO: 25 - DNA, 100 bp, Homo sapiens
gtgttcatgaaaaaaatgcacaaaatcctgcctggccggaataattcatgaagaaggggctggatccgtgggtcagagaacacaggaccagtttgccatc

SEQ ID NO: 26 - DNA, 100 bp, Homo sapiens
cattgctatgtccaccacaggggacaagattcttgatactgcactctctaagattggagagaaaagcctgtttaccaaggagcttgaacatgccctggag

SEQ ID NO: 27 - DNA, 100 bp, Homo sapiens
ggaaacttgggaatcattctggcttaaacaacacctcctcctgctgctcactcccgctgagcccactctactgccccagctccgtttctaccaccgcatc

SEQ ID NO: 28 - DNA, 100 bp, Homo sapiens
ggatcatgacctcgtgttctggttcggggacctgaacttccgcattgagagctatgacctgcactttgtcaagtttgccatcgacagtgaccagctccat

SEQ ID NO: 29 - DNA, 100 bp, Homo sapiens
cagagggaggtgttcctcttcaatgacctgctggtgattctcaaactttgcccgaagaagaagagctcctccacgtacaccttttgcaagtcagttggcc

SEQ ID NO: 30 - DNA, 100 bp, Homo sapiens
tggaactaaaagcagtgaagcaagggaggcaatcacactgaagcgggtcttcctccaggaacggggtcccacaggcgtgttgttttaaataacctgatgc

SEQ ID NO: 31 - DNA, 100 bp, Homo sapiens
catactcgggggacctttacaaagtgccgtcacctttgacctgtctggctcacactaggactgtcaccactctagctgtctgtatctctcctgaccatga

SEQ ID NO: 32 - DNA, 100 bp, Homo sapiens
ttcctcaccttgagcgggagtaacctgaccgtgaaggttgcatataacagctcaggaagctgtgagatagagaagatcgtgggctcagaaatagacagta

SEQ ID NO: 33 - DNA, 100 bp, Homo sapiens
tgatgattccatgactcccacctatgcagccttaaagccaaatccgcgtgtgtgtgtttgtgtctgtctgtgggtctcgaaggtgatccgtcggtgcggt

SEQ ID NO: 34 - DNA, 100 bp, Homo sapiens
atctcctccactgacttcctgagccacccttttgggatagctgtgtttgaggacaaggtgttctggacagacctggagaacgaggccattttcagtgcaa

SEQ ID NO: 35 - DNA, 100 bp, Homo sapiens
acagtgtccagatcaacagccatgtggtgcacaggtcggatggtaaatttcagatctttgcctatagagggaaagttcctgtggttgtgagttacagacc

SEQ ID NO: 36 - DNA, 100 bp, Homo sapiens
ctccttgagggaggagattcagcccggcgatattgtcattattgatcagttcattgacaggaccactatgagacctcagtccttctatgatggaagtcat

SEQ ID NO: 37 - DNA, 100 bp, Homo sapiens
tggaggtgttggccccatgacagtggcaatgctaatgaagaataccattattgctgcaaaaaaggtgctgaggcttgaagagcgagaagtgctgaagtct

SEQ ID NO: 38 - DNA, 100 bp, Homo sapiens
cattccttctgaggaggagatttttgcacctgaaaaaggcagccatagttttccagaagcaactcagaggtcagattgctcggagagtttacagacaatt

SEQ ID NO: 39 - DNA, 100 bp, Homo sapiens
tgttcatgcccatcactgcaatgacgttcagagtgagctgaaatacaaagctgaacatgtgaagcaaaaaggtcattatgttggtgtcccgacgatgaga

SEQ ID NO: 40 - DNA, 100 bp, Homo sapiens
cttgtggtttcaccctgaggaactggtagattacacgagctgtgctcagaactggatctatgaatgacaggagggcagaccacattgcttttcacatcca

SEQ ID NO: 41 - DNA, 100 bp, Homo sapiens
acagccgtggttttctgattttcaccatgcccggggcctcccttcccacctgcctgtgagaattggaggttagtgcctgaagctcagagctacacatttt

SEQ ID NO: 42 - DNA, 100 bp, Homo sapiens
gttttatttagtccagatgctgaaggatgggaagacccaagcagctggtgtggaggtctttcccagtgactcatcagtcattcagaaatcaaagcctgca

SEQ ID NO: 43 - DNA, 100 bp, Homo sapiens
gtgcacaaagaaaaaccctcatttccttaccaagaccggccctccacccaggagagcttccgccagaaactggctgcctttgctgggaccacatctggct

SEQ ID NO: 44 - DNA, 100 bp, Homo sapiens
gcattagcagttttgggtaagctggcggtactataacacgtactggaaacctgttcctcatcaccacctaccagattctggaaatgccgtcttctagaaa

SEQ ID NO: 45 - DNA, 100 bp, Homo sapiens
cgtggcaggatgactatactcagtatgtgatggaccaggaacttgcagacctcccgaaaacctacctgaggcgtcctgaagcatccagcccagccaggcc

SEQ ID NO: 46 - DNA, 100 bp, Homo sapiens
tggatgaaataggcgccgacattgaggacagacacattgtggtgtcctgcgcggccggcgtcaccatcagctccattgagaagaagctgtcagcgtttcg

SEQ ID NO: 47 - DNA, 100 bp, Homo sapiens
aaacagcctgtaggttttgtcagttttgacagtcgctcagaagcagaggctgcaaagaatgctttgaatggcatccgcttcgatcctgaaattccgcaaa

SEQ ID NO: 48 - DNA, 100 bp, Homo sapiens
atatttggctcagaacaggtgtccatgggacaaaaaagaacgatcctccacttgaccaagaaaaaagtgattctcccagaagcacaaagcatactcttgc

SEQ ID NO: 49 - DNA, 100 bp, Homo sapiens
cctttttggtggagaaaatggattggagtatcgaagcagcagttgctacttttgcccaagccagaccaccaggaatctacaagggtgattatttgaagga

SEQ ID NO: 50 - DNA, 100 bp, Homo sapiens
ccctgaaacacaagcaagtagatgaagaaaagatgtatgagaatgttcttaatgagtcgccagttcaattaccgcctctgccaccgaggaattggccttc

SEQ ID NO: 51 - DNA, 100 bp, Homo sapiens
attggcgctctcataggcctgtccgtagcagcagtggttcttctcgccttcattgttaccgcctgtgtgctctgctacctgttcatcagctctaagcccc

SEQ ID NO: 52 - DNA, 100 bp, Homo sapiens
tggtggctgcttgtcaggctgtggcttgattgcagcttctttctgtaacaccgtacagcaactatacgtctgtattggagtcattggaggtcttgggctt

SEQ ID NO: 53 - DNA, 100 bp, Homo sapiens
ggcttggtagtgtttgccatcgtctttggtgtggcgctgcggaagctggggcctgaaggggagctgcttatccgcttcttcaactccttcaatgaggcca

SEQ ID NO: 54 - DNA, 100 bp, Homo sapiens
agcttgctgaaggctgccctctccacaggcttcatgttcttctcgtatgaattcttctgtaatgtcttccactgcatgaacaggacagccagccagcgct

SEQ ID NO: 55 - DNA, 100 bp, Homo sapiens
tagcagtacttccttcctgactgtgactcctactacctgccagccttcttccttgctctgcgctgggatcatactcccaaatcacattactaaatgccaa

SEQ ID NO: 56 - DNA, 100 bp, Homo sapiens
gcagcgtggggccgagaggaatagtgagcgatggcggaaaacctgaaaagactggtctcaaacgaaactttacgaacgttgcaggaaaagctagacttct

SEQ ID NO: 57 - DNA, 100 bp, Homo sapiens
taagcttaaaggggaaccatttgtgaatgaatatttggaacttaccaagtcctaagagacttttggaagaggatatatatagcatagtaccataccactt

SEQ ID NO: 58 - DNA, 100 bp, Homo sapiens
aacagggtcacccacatcttgaacatggcccgggagattgacaacttctaccctgagcgcttcacctaccacaatgtgcgcctctgggatgaggagtcgg

SEQ ID NO: 59 - DNA, 100 bp, Homo sapiens
cagtttccaccatctcggtcatcaggattgcctaatatacctgtccagacaatctccagagctgctgcagaaaagctgtttgggaatatggaaggagact

SEQ ID NO: 60 - DNA, 100 bp, Homo sapiens
accgctgtgtccgggtgggtggtcagaatgccgtgctccaggtgttcacagctgcttcgtggaagaccatgtgctccgatgactggaagggtcactacgc

SEQ ID NO: 61 - DNA, 100 bp, Homo sapiens
gggcacattcgaggagtgccaccggaagtgcaaggagctgtttcccattcagatggagggtgtcaagctcacagtcaacaaagggttgagtaaccatttt

SEQ ID NO: 62 - DNA, 100 bp, Homo sapiens
tctggtctggccttcgtgttggcttcaaggtggtaacagagagagctgcagagtgggggtgtgtttgccagtccctgcatgggtgaatgttggggtgccc

SEQ ID NO: 63 - DNA, 100 bp, Homo sapiens
aagagacagaaaacataattgcagcaaatcactgggttctacctgcagctgaattccatgggctttgggacagcttggtatacgatgtggaagtcaaatc

SEQ ID NO: 64 - DNA, 100 bp, Homo sapiens
ctatctttcttgtagcaaagctgcacctgatgatgctgcctctcctctctgtgttgtctgggcccttgtttacaagcacgcgttacccttcctgagggga

SEQ ID NO: 65 - DNA, 100 bp, Homo sapiens
tgacgggtcgcgtcagttccgacccggacccgtacgctgctgcgctgacgtggctcctggaagcagggctggcgtagggccgccatgttgcagcaggata

SEQ ID NO: 66 - DNA, 100 bp, Homo sapiens
cccagaaatagacctctcctgtagagtggtgatatacagaatgagtttcagtttgcattgcagctgggattgaaagtaatcagtcgtgagcaggcaggca

SEQ ID NO: 67 - DNA, 100 bp, Homo sapiens
tgctggagggccaggagccggacggggtgcgctttgaccgcgagagggcgcgccgcctgtgggaagccgtgtccggtgcccagccggtgggtagagagga

SEQ ID NO: 68 - DNA, 100 bp, Homo sapiens
tcacacgtgctctgttctcggggttgttccattcatgccttcttggagggtgagggtggcttgggaaccgacccagtgatcatgcctactttcttctttg

SEQ ID NO: 69 - DNA, 100 bp, Homo sapiens
tgttcagacacatgaaagggctcatagtggaaggaaactctatgtttgtgaggaatgcggaaaaacatttatttcccattcaaaccttcaaagacacagg

(19) United States
(12) Patent Application Publication
(10) Pub. No.: US 2020/0299783 A1
(43) Pub. Date: Sep. 24, 2020

(54) MOLECULAR SIGNATURE FOR SELECTING LYMPHOMA PATIENTS FOR TREATMENT WITH IBRUTINIB

(71) Applicant: Board of Regents, The University of Texas System, Austin, TX (US)

(72) Inventors: Michael WANG, Houston, TX (US); Linghua WANG, Houston, TX (US); Shaojun ZHANG, Houston, TX (US); Liang ZHANG, Houston, TX (US); Krystle NOMIE, Houston, TX (US); Makhdum AHMED, Houston, TX (US); Yixin YAO, Houston, TX (US)

(21) Appl. No.: 16/821,638

(22) Filed: Mar. 17, 2020

Related U.S. Application Data
(60) Provisional application No. 62/819,937, filed on Mar. 18, 2019.

(51) Int. Cl. C12Q 1/6886; G01N 33/68; A61K 35/17; A61K 31/519; A61K 31/496

(52) U.S. Cl. CPC C12Q 1/6886; G01N 33/68; A61K 35/17; A61K 31/519; G01N 2800/7028; C12Q 2600/158; C12Q 2600/106; G01N 2800/52; A61K 31/496

(57) ABSTRACT

Provided herein is a molecular signature that delineates response to ibrutinib and its use in predicting responsiveness to ibrutinib for lymphoma patients, and in particular for mantle cell lymphoma patients. Also provided are methods of treating a patient that is predicted to be ibrutinib-resistant with an inhibitor of oxidative phosphorylation, a BH3-mimetic, a noncovalent BTK inhibitor, or a CAR-T therapy.

### REFERENCE TO RELATED APPLICATIONS

The present application claims the priority benefit of U.S. provisional application No. 62/819,937, filed Mar. 18, 2019, the entire contents of which are incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on Mar. 17, 2020, is named UTSCP1441US_ST25.txt and is 16 kilobytes in size.

### BACKGROUND

### 1. Field

The present invention relates generally to the fields of medicine and oncology. More particularly, it concerns compositions and methods for classifying lymphoma patients as being sensitive or resistant to ibrutinib as well as using such classification in treating lymphoma patients.

### 2. Description of Related Art

Mantle cell lymphoma (MCL) is a subtype of B-cell lymphoma that accounts for 6%-8% of all non-Hodgkin lymphoma cases (Fernandez et al., 2005; Perez-Galan et al., 2011). Constitutive activation of the B-cell receptor (BCR) pathway is a hallmark of B-cell lymphomas, including MCL (Kuppers, 2005; Saba et al., 2016). Bruton's tyrosine kinase (BTK) is a critical component of the BCR pathway that is highly expressed in MCL cells, including its active phosphorylated form (Myklebust et al., 2017). Ibrutinib, a first-in-class oral covalent inhibitor of BTK, was approved by the US Food and Drug Administration (FDA) in 2013 to treat relapsed/refractory MCL, and this kinase inhibitor has demonstrated significant and efficacious anti-tumor activity with an overall response rate of 68% in relapsed/refractory MCL and median duration of response of approximately 18 months (Wang et al., 2013). However, once patients relapse after ibrutinib treatment, the 1-year survival rate is only 22% (Cheah et al., 2015), prompting an urgent need to identify alternative therapeutic options that will benefit this patient population.

The mechanisms mediating ibrutinib resistance have recently been explored. The presence of a relapse-specific C481S mutation in the BTK gene and PLCG2 mutations associated with acquired ibrutinib resistance have been identified in multiple chronic lymphocytic leukemia (CLL) clinical specimens, suggesting that the potential silencing of the BCR-BTK pathway likely plays a role in ibrutinib resistance (Woyack, 2015; Woyach et al., 2017). However, BTK and PLCG2 mutations are infrequent in MCL patients (Jain et al., 2018). On the other hand, the activation of the PI3K-AKT-mTOR pathway and alternative NF-κB signaling have been implicated as alternative survival mechanisms that override the effects of ibrutinib, initiating resistance (Chiron et al., 2014; Rahal et al., 2014; Zhaeo et al., 2017). Nevertheless, the mechanisms underlying ibrutinib resistance are most likely complicated, and studies fully elucidating the intricate networks responsible for therapeutic resistance are still lacking.

Although inhibition of the PI3K-AKT-mTOR pathway in preclinical models of MCL has shown strong anti-tumor activity in vitro (Lannutti et al., 2011; Tabe et al., 2014), this inhibition has not resulted in significant effects in vivo using MCL PDX mouse models (Zhaeo et al., 2017; Zhang et al., 2017). Indeed, PI3K inhibitors such as CAL-101 (idelalisib) (Kahl et al., 2014) and several rapamycin analogs (everolimus and temsirolimus) (Lorenzi et al., 2014; Hess et al., 2009; Calimberti & Petrini, 2010; Witzig et al., 2005; Yee et al., 2006) have resulted in underwhelming clinical outcomes in MCL and myriad other cancers, suggesting that novel inhibitors and/or alternative therapeutic targets are needed.

### SUMMARY

Provided herein are a gene set and customized list of gene probes for use in classifying patients with lymphoma as being either sensitive or resistant to ibrutinib. These can be used to select subsets of patients for administration of either ibrutinib or an oxidative phosphorylation inhibitor if the patient is identified as being sensitive or resistant to ibrutinib, respectively.

In one embodiment, provided herein are methods of classifying a patient having a lymphoma as being either sensitive or resistant to ibrutinib, the methods comprising: (a) obtaining a sample of the patient's lymphoma; (b) measuring an expression level of a plurality of genes in the sample, wherein each gene in the plurality of genes is selected from Table 1; (c) generating an expression profile based on a comparison between the expression level of the plurality of genes in the sample and a corresponding expression level obtained from a reference sample; and (d) classifying the patient as having a lymphoma that is either sensitive or resistant to ibrutinib based on the expression profile.

In one embodiment, provided herein are methods of treating a patient having a lymphoma, the methods comprising: (a) detecting whether the patient's cancer has an ibrutinib-sensitive or ibrutinib-resistant gene expression profile relative to a reference sample by: (i) obtaining or having obtained a sample of the patient's lymphoma; (ii) performing or having performed an assay on the sample to measure an expression level of a plurality of genes in the sample, wherein each gene in the plurality of genes is selected from Table 1; and (iii) generating an expression profile based on a comparison between the expression level of the plurality of genes in the sample and a corresponding expression level obtained from a reference sample; (b) selecting or having selected a treatment for the patient based on whether the patient's cancer has an ibrutinib-sensitive or ibrutinib-resistant gene expression profile; and (c) administering or having administered to the patient a therapeutically effective amount of: (i) ibrutinib, if the patient is determined to have an ibrutinib-sensitive lymphoma; or (ii) an inhibitor of oxidative phosphorylation, a BH3-mimetic, a noncovalent BTK inhibitor, or a CAR-T therapy, if the patient is determined to have an ibrutinib-resistant lymphoma. In other words, provided herein are methods of treating a patient having a lymphoma, the methods comprising administering a therapeutically effective amount of ibrutinib to the patient, wherein the patient's cancer has been determined to have an ibrutinib-sensitive gene expression profile as determined by assaying the expression level of a plurality of genes selected from Table 1. Alternatively, provided herein are methods of treating a patient having a lymphoma, the methods comprising administering a therapeutically effective amount of an inhibitor of oxidative phosphorylation, a BH3-mimetic, a noncovalent BTK inhibitor, or a CAR-T therapy to the patient, wherein the patient's cancer has been determined to have an ibrutinib-resistant gene expression profile as determined by assaying the expression level of a plurality of genes selected from Table 1

In some aspects, the plurality of genes comprises at least 40 genes selected from Table 1. In some aspects, the plurality of genes comprises at least 45, 50, 55, or 60 genes selected from Table 1. In some aspects, the plurality of genes comprises all 69 genes selected from Table 1.

In some aspects, the patient is classified as having an ibrutinib-resistant lymphoma if the expression of SLC16A1 is upregulated. In some aspects, the patient is classified as having an ibrutinib-resistant lymphoma if the expression of at least two of the following genes is upregulated: HR, HMBS, HN1, PYCR1, SLC26A8, SEPT3, INPP5J, SLC1A5, CCDC86, CTPS1, TOMM40, TFRC, TRIP13, LRP8, SQLE, HIVEP3, FADS1, TTLL12, SLC25A19, RCC1, NPM3, CCT5, DDX21, MTHFD2, SLC16A1, and NME1. In some aspects, the patient is classified as having an ibrutinib-resistant lymphoma if the expression of at least two of the following genes is downregulated: LCN8, CD84, OR2C1, CD8A, COL4A4, COL4A3, MFHAS1, SSH3, MYO10, PTPRN2, SPATA18, PCSK1, BAZ2B, PSO3, NEB, PLCXD2, ZNF433, ADAMTS10, ARMCX4, TVP23C, ACMSD, AGRP, IQSEC3, RNGTT, FAAH2, CCDC173, SCIMP, PRAG1, TOR4A, ZNF395, RBPMS, BFSP2, LDLRAD4, A4FALT, KBTBD6, FAM159A, and ARSD. In some aspects, the patient is classified as having an ibrutinib-resistant lymphoma if: (a) the expression of at least two of the following genes is upregulated: HR, HMBS, HN1, PYCR1, SLC26A8, SEPT3, INPP5J, SLC1A5, CCDC86, CTPS1, TOMM40, TFRC, TRIP13, LRP8, SQLE, HIVEP3, FADS1, TTLL12, SLC25A19, RCC1, NPM3, CCT5, DDX21, MTHFD2, SLC16A1, and NME1; and (b) the expression of at least two of the following genes is downregulated: LCN8, CD84, OR2C1, CD8A, COL4A4, COL4A3, MFHAS1, SSH3, MYO10, PTPRN2, SPATA18, PCSK1, BAZ2B, PSO3, NEB, PLCXD2, ZNF433, ADAMTS10, ARMCX4, TVP23C, ACMSD, AGRP, IQSEC3, RNGTT, FAAH2, CCDC173, SCIMP, PRAG1, TOR4A, ZNF395, RBPMS, BFSP2, LDLRAD4, A4FALT, KBTBD6, FAM159A, and ARSD.

In some aspects, the patient is classified as having an ibrutinib-sensitive lymphoma if the expression of at least two of the following genes is downregulated: HR, HMBS, HN1, PYCR1, SLC26A8, SEPT3, INPP5J, SLC1A5, CCDC86, CTPS1, TOMM40, TFRC, TRIP13, LRP8, SQLE, HIVEP3, FADS1, TTLL12, SLC25A19, RCC1, NPM3, CCT5, DDX21, MTHFD2, SLC16A1, and NME1. In some aspects, the patient is classified as having an ibrutinib-sensitive lymphoma if the expression of at least two of the following genes is upregulated: LCN8, CD84, OR2C1, CD8A, COL4A4, COL4A3, MFHAS1, SSH3, MYO10, PTPRN2, SPATA18, PCSK1, BAZ2B, PSO3, NEB, PLCXD2, ZNF433, ADAMTS10, ARMCX4, TVP23C, ACMSD, AGRP, IQ5EC3, RNGTT, FAAH2, CCDC173, SCIMP, PRAG1, TOR4A, ZNF395, RBPMS, BFSP2, LDLRAD4, A4FALT, KBTBD6, FAM159A, and ARSD. In some aspects, the patient is classified as having an ibrutinib-sensitive lymphoma if: (a) the expression of at least two of the following genes is downregulated: HR, HMBS, HN1, PYCR1, SLC26A8, SEPT3, INPP5J, SLC1A5, CCDC86, CTPS1, TOMM40, TFRC, TRIP13, LRP8, SQLE, HIVEP3, FADS1, TTLL12, SLC25A19, RCC1, NPM3, CCT5, DDX21, MTHFD2, SLC16A1, and NME1; (b) the expression of at least two of the following genes is upregulated: LCN8, CD84, OR2C1, CD8A, COL4A4, COL4A3, MFHAS1, SSH3, MYO10, PTPRN2, SPATA18, PCSK1, BAZ2B, PSO3, NEB, PLCXD2, ZNF433, ADAMTS10, ARMCX4, TVP23C, ACMSD, AGRP, IQSEC3, RNGTT, FAAH2, CCDC173, SCIMP, PRAG1, TOR4A, ZNF395, RBPMS, BFSP2, LDLRAD4, A4FALT, KBTBD6, FAM159A, and ARSD.

In some aspects, the expression level of the plurality of genes is measured by detecting a level of mRNA transcribed from the plurality of genes. In certain aspects, the mRNA level is detected by microarray, RT-PCR, qRT-PCR, nanostring assay, or in situ hybridization. In certain aspects, the mRNA level is measured using nanostring probes. In certain aspects, the nanostring probes hybridize to the target sequence listed in Table 1 for each of the plurality of genes.

In some aspects, the expression level of the plurality of genes is measured by detecting a level of cDNA produced from reverse transcription of mRNA transcribed from the plurality of genes. In some aspects, the expression level of the plurality of genes is measured by detecting a level of polypeptide encoded by the plurality of genes.

In some aspects, the sample is a formalin-fixed, paraffin-embedded sample. In some aspects, the sample is a fresh frozen sample.

In some aspects, the reference sample is a sample from a healthy subject or a group of healthy subjects. In some aspects, the reference sample is a sample of non-cancerous cells obtained from the patient.

In some aspects, the methods further comprise reporting the classification of the patient. In some aspects, the reporting comprises preparing a written or electronic report. In certain aspects, the methods further comprise providing the report to the patient, a doctor, a hospital, or an insurance company.

In some aspects, if the patient is determined to have an ibrutinib-resistant lymphoma, then the method further comprises administering a therapeutically effective amount of an inhibitor of oxidative phosphorylation to the patient. In certain aspects, the inhibitor of oxidative phosphorylation is IACS-010759 (CAS [1570496-34-2]). In certain aspects, the BH3-mimetic is venetoclax (ABT-199). In certain aspects, the noncovalent BTK inhibitor is Loxo-305. In certain aspects, the CAR-T therapy targets CD19. In certain aspects, the methods further comprise administering a therapeutically effective amount of a glutaminase inhibitor to the patient. In certain aspects, the methods further comprise administering a therapeutically effective amount of an mTOR inhibitor to the patient.

In some aspects, if the patient is determined to have an ibrutinib-sensitive lymphoma, then the method further comprises administering a therapeutically effective amount of ibrutinib to the patient.

In some aspects, the lymphoma is mantle cell lymphoma. In some aspects, the patient has previously failed to respond to treatment with ibrutinib. In some aspects, the patient is previously been treated with ibrutinib. In certain aspects, the patient has relapsed following treatment with ibrutinib.

In one embodiment, provided herein are compositions comprising a set of nanostring probes that hybridize to the target sequence for at least 40 of the genes listed in Table 1. In some aspects, the composition comprises nanostring probes that hybridize to the target sequence for at least 45, 50, 55, or 60 of the genes listed in Table 1. In some aspects, the composition comprises nanostring probes that hybridize to the target sequence for all 69 of the genes listed in Table 1.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-C. Landscape of somatic mutations and DNA copy number alterations in 14 MCL patients treated with ibrutinib. (FIG. 1A) Somatic mutations. Each column represents a patient tumor sample and the clinical and pathological characteristics were as annotated at the top. Genes with nonsynonymous mutations or copy number alterations in two or more patients were listed. The numbers on the left and right sides represent the percentage of MCL tumors carrying mutation or copy number alteration of each specific gene in the ibrutinib-sensitive and -resistant groups, respectively. MIPI score, 0-3, low risk; 4-5, intermediate risk; 6-11, high risk; CR, complete response; PR, partial response; PD, progression disease; LOH, loss of heterozygosity. (FIG. 1B) Somatic copy number alterations in ibrutinib-resistant (top) and -sensitive (bottom) tumors. The chromosome numbers are labeled at the top, and the sample IDs are shown on the left. Blue indicates copy number loss, and red indicates copy number gain where the intensity corresponds to the log 2Ratio of each segment. (FIG. 1C) Copy number gains and losses in ibrutinib-sensitive (blue) and -resistant (pink) groups. Box in the box plot represents the inter-quartile range (IQR) where the centerline depicts the median. The upper whisker indicates the maximum value or 75^{th} percentile+1.5 IQR, whichever is smaller; the lower whisker indicates the minimum value or 25^{th} percentile −1.5 IQR, whichever is greater.

FIGS. 2A-D. DEGs between the ibrutinib-sensitive and -resistant tumors. (FIG. 2A) Unsupervised clustering showing the most differentially expressed gene (DEGs) between the sensitive and resistant tumors. (FIG. 2B) A cutoff of fold change ≥2 or ≤−2 and the FDR q-value ≤0.01 was applied, and only genes that met these criteria were selected for unsupervised clustering in FIG. 2A and labeled in the volcano plot. (FIG. 2C) The box plots showing two representative DEGs of metabolite transporters SLC16A1 and SLC1A5. (FIG. 2D) Immunoblotting showing the differential expression of the metabolite transporters SLC15A1 and SLC1A5 in ibrutinib-resistant MCL cell lines Maver-1 and Z-138 and the ibrutinib-sensitive MCL cell lines JeKo-1 and Mino.

FIGS. 3A-B. Significantly enriched signaling pathways in the ibrutinib-resistant tumors. (FIG. 3A) The most significantly enriched pathways by GSEA analysis. The listed pathways are ranked by their normalized enrichment scores and colored by their type classification. The FDR q-values are labeled on the right. (FIG. 3B) Representative enrichment plots for the hallmark OXPHOS pathway, mTORC1 signaling, and MYC and E2F targets (Zhang et al., 2019).

FIGS. 4A-E. Ibrutinib-resistant MCL cells rely on OXPHOS for energy production. (FIG. 4A) The basal OCR level and (FIG. 4B) ATP-coupled OCR in ibrutinib-sensitive (JeKo-1 and Mino) and ibrutinib-resistant (Z-138 and Maver-1) MCL cell lines (***, p<0.0001; mixed effects regression model) (n=3 biological replicates; mean±SD). (FIG. 4C) Relative abundance of metabolites extracted from ibrutinib-sensitive and ibrutinib-resistant MCL cell lines. Each row represents a single metabolite and each column represents an MCL cell line (from left to right: JeKo-1, Mino, JVM-1, JeKo-1 BTK KD, Maver-1, and JVM-13). (FIG. 4D) RPPA heatmap showing the differential protein expression between in ibrutinib-sensitive and ibrutinib-resistant MCL cell lines (from left to right in triplicate: Rec-1, Mino, Z-138, and Mayer-1). (FIG. 4E) ROS levels detected by flow cytometry in MCL cell lines as indicated in the absence of presence of 2 mM glutamine.

FIGS. 5A-G. Targeting the OXPHOS pathway to overcome ibrutinib resistance. (FIG. 5A) Cell growth inhibition of ibrutinib-resistant (Z-138 and Maver-1) and ibrutinib-sensitive (Mino and JeKo-1) MCL cell lines after 72-hour incubation with IACS-010759 at the indicated doses (beta regression model; p<0.0001; n=3 biological replicates, mean±SEM). (FIG. 5B) The mitochondria OCR quantitated in ibrutinib-resistant (Z-138 and Maver-1) and ibrutinib-sensitive (Mino) MCL cell lines treated with 20 nM IACS-010759 for 1 h (mixed effects regression model; p<0.0001 between the 2 resistant cell lines and the sensitive cell line; n=3 biological replicates, mean±SEM). No significant difference observed between the control and IACS-010759 treatment in the Mino cell line (mixed effects regression model; p=0.0832). IACS-010759 produced an average reduction of 358.9406 (95% CI: 321.1402-396.741) OCR compared with the control; mixed effects regression model; p<0.0001. (FIG. 5C) Mitochondria membrane potential (Δψm) depicted as histograms in the indicated MCL cell lines treated with 20 nM IACS-010759 and/or 5 μM BPTES for 24 hours before staining with TMRE dye. (FIG. 5D) Relative Δψm shown as % TMRE high-intensity cell counts in C (n=3 biological replicates, mean±SD). Synergistic and additive effects were observed after combination treatment in the ibrutinib-resistant Z-138 and Maver-1 cell lines (p<0.0001) but not for the ibrutinib-sensitive Mino and JeKo-1 cell lines (beta regression model; p=0.0757). The Mino and JeKo-1 cell lines had higher % TMRE high-intensity cell counts compared with the Z-138 and Maver-1 lines after combination treatment (beta regression model; p<0.0001). (FIG. 5E) ROS levels measured in ibrutinib-sensitive and -resistant MCL cell lines treated with 20 nM IACS-010759 and/or 5 μM BPTES for 24 hours. (FIG. 5F) Quantitative analysis of Z-138 and Maver-1 apoptotic cells (n=3 biological replicates, mean±SD). Single agent treatment compared with the control. p calculated by two-sample t-test after logistic transformation. (FIG. 5G) Immunoblotting of cCaspase-7 and cPARP (cleaved) and full-length Caspase-7 and PARP in MCL cell lines as listed in F, treated with 20 nM IACS-010759 and/or 5 μM BPTES for 72 hours.

FIGS. 6A-D. The anti-MCL activity of IACS-010759 in an ibrutinib-resistant PDX model. The mice were administered vehicle control, ibrutinib (50 mg/kg oral gavage, daily), or IACS-010759 (10 mg/kg oral gavage, 5 consecutive days/week) beginning 5 days after engraftment until the endpoint. (FIG. 6A) Tumor volume calculated to reflect tumor burden (n=5; ibrutinib vs vehicle p=0.6397, vehicle vs IACS-010759 p<0.0001, mixed effects regression model after logarithmic transformation) as indicated. (FIG. 6B) Human β₂M levels to monitor tumor burden (vehicle vs IACS-010759, mixed effects regression model after logarithmic transformation; p<0.0001) on Days 0, 10 and 20 of treatment. (FIG. 6C) Mouse body weight calculated during drug treatment (vehicle vs IACS-010759, mixed effects regression model after logarithmic transformation; p=0.3304; mean±SEM). (FIG. 6D) H&E and CD20 staining of representative mouse tumors dissected at the end of treatment.

FIGS. 7A-B. Survival outcomes of MCL patients relative to ibrutinib response. (FIG. 7A) Kaplan-Meier overall survival and (FIG. 7B) progression-free survival curves showing the significant clinical outcomes between ibrutinib-sensitive (n=19) and -resistant patients (n=9). p<0.0001.

FIGS. 8A-B. Tumor cellularity comparison between the ibrutinib-sensitive and -resistant clinical specimens. (FIG. 8A) The pathological tumor cellularity estimated by the percentage of CD19+ malignant cells Box in the box plot represents the inter-quartile range (IQR) where the centerline depicts the median. The upper whisker indicates the maximum value or 75th percentile+1.5 IQR, whichever is smaller; the lower whisker indicates the minimum value or 25th percentile −1.5 IQR, whichever is greater. (FIG. 8B) The tumor cellularity reflected by the distribution of the tumor variant allelic fraction of all somatic mutations identified from the most frequently mutated genes in MCL. N.S., not statistically significant.

FIGS. 9A-B. DEGs between the ibrutinib-sensitive and -resistant tumors using the nanoString nCounter system. (FIG. 9A) Heat map showing differentially expressed genes between BTK-inhibitor sensitive (N=11) versus-resistant (N=5) patient samples. Genes belonging to the DNA damage repair, chromatin modification and cell cycle, and apoptosis pathways were most differentially expressed in the resistant group demonstrating significantly higher expression of DNA damage repair genes. (FIG. 9B) Pathway enrichment signature scores for DNA damage repair showing resistant samples have significantly higher scores compared with the sensitive samples (p=0.031). Box in the box plot represents the inter-quartile range (IQR) where the centerline depicts the median. The upper whisker indicates the maximum value or 75th percentile +1.5 IQR, whichever is smaller; the lower whisker indicates the minimum value or 25th percentile −1.5 IQR, whichever is greater.

FIGS. 10A-D. OXPHOS pathway is active in ibrutinib-resistant MCL cells. (FIG. 10A) Glutamine uptake in the ibrutinib-sensitive MCL cell lines JeKo-1 and Mino and the ibrutinib-resistant MCL cell lines Maver-1 and Z-138 (sensitive lines vs resistant lines; mixed effects regression model; p=0.0304; n=3 biological replicates, mean±SD). (FIG. 10B) ROS levels determined by flow cytometry in CM-H2DCF-DA-stained MCL cell lines treated with 0.5 mM AOA for 24 hours. (FIG. 10C) Immunoblotting of BTK in the parental JeKo-1 wildtype cell line compared with two BTK knockdown clones (KD1 and KD2). (FIG. 10D) Ingenuity Pathway Analysis (IPA) showing OXPHOS pathway enrichment in JeKo-1 BTK-KD1 and BTK-KD2 clones compared with the parental JeKo-1 cell line. The numbers to the right of each bar represent the number of proteins belonging the denoted pathway.

FIGS. 11A-E. Inhibition of glutaminolysis or OXPHOS reduces mitochondria activity and ATP production and induces apoptosis in ibrutinib-resistant MCL cells. (FIG. 11A) ETC 1 complex activity in the ibrutinib-resistant Z-138 and Maver-1 MCL cell lines treated with 20 nM IACS-0101759 for 24 hours (linear regression model; 0 nM vs 20 nM: p=0.0041; n=3 biological replicates, mean±SD). (FIG. 11B) MitoTracker and Hoechst staining of Maver-1 and Z-138 MCL cell lines after treatment with 20 nM IACS-010759 for 24 hours (representative cells are shown). (FIG. 11C) Intracellular ATP levels in ibrutinib-sensitive (JeKo-1 and Rec-1; 0 nM vs 20 nM; linear regression model: p=0.9504) and ibrutinib-resistant (Maver-1 and Z-138; 0 nM vs 20 nM; linear regression model: p<0.0001) MCL cell lines treated with 20 nM of IACS-010759 for 24 hours (n=3 biological replicates, mean±SD). (FIG. 11D) Glutamine uptake in ibrutinib-sensitive MCL cell lines (JeKo-1 and Mino) and ibrutinib-resistant MCL cell lines (Maver-1 and Z-138) treated with 20 nM IACS-010759 for 24 hours (mixed effects regression model; p=0.0071; n=3 biological replicates, mean±SD). (FIG. 11E) Annexin-V/PI staining and flow cytometry of ibrutinib-sensitive (Rec-1 and Mino) and ibrutinib-resistant (Z-138 and Maver-1) MCL cell lines treated with 20 nM IACS-010759 and/or 5 μM BPTES for 72 hours.

FIGS. 12A-D. The anti-cancer effects of IACS-010759 in an ibrutinib-resistant high-grade B-cell lymphoma PDX model. The mice were administered vehicle control, ibrutinib (50 mg/kg oral gavage, daily), or IACS-010759 (10 mg/kg oral gavage, 5 consecutive days/week) beginning 5 days after engraftment until the endpoint. (FIG. 12A) Tumor volume calculated to reflect tumor burden (n=5; ibrutinib vs vehicle p=0.7227, vehicle vs IACS-010759 p<0.0001, mixed effects regression model after logarithmic transformation) and (FIG. 12B) Human β₂M levels to monitor tumor burden on Days 0, 7 and 15 of treatment (n=5; ibrutinib vs vehicle, p=0.6911, IACS-010759 vs vehicle, p=0.0003, mixed effects regression model after logarithmic transformation). (FIG. 12C) Mouse body weight calculated during drug treatment (IACS-010759 vs vehicle, mixed effects regression model after logarithmic transformation, p=0.1964). (FIG. 12D) Survival curve of the ibrutinib-resistant PDX model (n=5; IACS-010759 vs vehicle, p=0.0035, log-rank test).

### DETAILED DESCRIPTION

Mantle cell lymphoma (MCL) accounts for 6%-8% of all non-Hodgkin's lymphoma cases and is a rare and incurable B-cell lymphoma subtype. Constitutive activation of the B-cell receptor pathway is a hallmark of B-cell lymphomas, including MCL. Bruton's tyrosine kinase (BTK) is a critical component of the B-cell receptor pathway, and ibrutinib, a first-in-class, once-daily, and oral covalent inhibitor of BTK, was developed to reduce/silence B-cell receptor pathway activity, leading to remarkable anti-tumor activity both in the laboratory and in the clinic. In a prior multiple-center Phase II clinical trial, the overall response rate in relapsed/refractory MCL patients was 68%, with a median progression free survival (PFS) of 13.9 months, surpassing the effectiveness of other therapies. Follow-up data demonstrate the durability of responses and confirm the unprecedented single-agent activity of ibrutinib in relapsed/refractory MCL. These results led to the FDA approval of ibrutinib to treat relapsed/refractory MCL in 2013 via the “breakthrough” mechanism.

Based on another Phase II clinical trial, approximately 43% of patients showed no response to ibrutinib or displayed initial positive responses but also experienced disease progression within 12 months of treatment. Furthermore, once patients relapse after ibrutinib treatment, the 1-year survival rate is only 22%, demonstrating that novel treatment strategies are urgently needed.

To further understand the mechanisms mediating ibrutinib resistance, in-depth RNA sequencing analysis of ibrutinib-sensitive and ibrutinib-resistant MCL clinical specimens was performed and a 63-gene molecular signature that clearly delineates between sensitivity and resistance was identified. A nanoString panel based on this molecular signature, which can be used in a clinical setting, has been developed (Table 1).

In addition, RNAseq data were analyzed and overregulated pathways associated with ibrutinib resistance were found. Inhibition of these dysregulated pathways using novel targeted agents produced dramatic growth inhibition and increased apoptosis of MCL cells. As such, targeting these pathways may overcome ibrutinib resistance in combination with other targeted therapeutics. For example, metabolic reprogramming occurs in ibrutinib-resistant MCL cells, producing a reliance on oxidative phosphorylation (OXPHOS) and glutaminolysis for growth and survival. Quenching OXPHOS energy production using a novel small molecule inhibitor (IACS-010759) (Molina et al., 2018) targeting complex I of the mitochondrial electron transport chain (ETC) resulted in decreased proliferation and increased apoptosis in ibrutinib-resistant patient-derived cancer models. Thus, targeting metabolic pathways to subvert therapeutic resistance is a clinically viable approach to treat highly refractory malignancies.

TABLE 1
69-gene molecular signature.

| | | | | SEQ ID |
|---|---|---|---|---|
| Gene name | Accession | Position | Target Sequence | NO: |
| A4GALT | NM_017436.4 | 799-898 | ATCGCACTCATGTGGAAGTTC | 1 |
| | | | GGCGGCATCTACCTGGACAC | |
| | | | GGACTTCATTGTTCTCAAGAA | |
| | | | CCTGCGGAACCTGACCAACGT | |
| | | | GCTGGGCACCCAGTCCC | |
| ACMSD | NM_138326.2 | 469-568 | AGAGCTGGGCTTTCCCGGGGT | 2 |
| | | | CCAAATTGGCACCCACGTCAA | |
| | | | CGAGTGGGACCTGAACGCGC | |
| | | | AGGAGCTCTTTCCTGTCTATG | |
| | | | CGGCAGCCGAAAGGCTG | |
| ADAMTS10 | NM_030957.3 | 2455- | CCTGGGGCCGGGTACGAGGA | 3 |
| | | 2554 | TGTCGTCTGGATTCCCAAAGG | |
| | | | CTCCGTCCACATCTTCATCCA | |
| | | | GGATCTGAACCTCTCTCTCAG | |
| | | | TCACTTGGCCCTGAAGG | |
| AGRP | NM_001138.1 | 18-117 | CCTGTGGAAATTTGTGGACCC | 4 |
| | | | TGGGCACCCTCTCTTGCTCCC | |
| | | | AAATTTTAATCGGCTCCTGGA | |
| | | | AACCTCACCCCAAATTGGAG | |
| | | | ATAGGCACTCCTCTTGT | |
| ARMCX4 | NR_045861.1 | 309-408 | GAAAGGAAGGAGAGGAGAA | 5 |
| | | | AACTGCACTGGATCAAGGAG | |
| | | | GTCTTCCCATAGGCCTGCAGT | |
| | | | AGGGCTGTTTACCATTGGAAG | |
| | | | CAAGGGAAAGAGGAGGAGA | |
| ARSD | NM_001669.3 | 209-308 | AATGCCTTTAAACCAAATATC | 6 |
| | | | CTACTGATCATGGCGGATGAT | |
| | | | CTAGGCACTGGGGATCTCGGT | |
| | | | TGCTACGGGAACAATACACT | |
| | | | GAGAACGCCGAATATTG | |
| BAZ2B | NM_013450.2 | 3901- | TATGGAAGCCCACTGTGGAC | 7 |
| | | 4000 | AAACTGAGCTTACTGAAAGTC | |
| | | | TGAAGACCAAAGCTTTTCAGG | |
| | | | CTCACACTCCAGCACAGAAA | |
| | | | GCTTCAGTCCTGGCTTTC | |
| BFSP2 | NM_003571.2 | 798-897 | AATGGACCTGGAGAGTCAAA | 8 |
| | | | TAGAAAGTCTGAAAGAAGAA | |
| | | | CTTGGCTCTCTATCAAGAAAC | |
| | | | TATGAAGAGGATGTGAAGCT | |
| | | | GCTGCACAAACAGTTGGCA | |
| CCDC173 | NM_001085447.1 | 195-294 | ATCTGCCTCTTCTACCTAGCA | 9 |
| | | | AAGTAGATCTCCAGCAGGTC | |
| | | | ACCATAATTCCACACGATGAG | |
| | | | TGGAAAAGGATTCAAGATAG | |
| | | | CCTTGACAGGTTGACAAG | |
| CCDC86 | NM_024098.3 | 1461- | AGAGATGGGGGCGGGAAGAG | 10 |
| | | 1560 | ATTCAGCTCCCATCCCTCCTT | |
| | | | CCTCTCCTTCTCCAAGTGCCT | |
| | | | TCAAACCAAGAACTGTACATT | |
| | | | CTTCTGGTTCCTCAGTG | |
| CCT5 | NM_012073.3 | 2178- | TCATATTGAGAGGAATATGG | 11 |
| | | 2277 | GCTTGATCCTCTTCCTATCTA | |
| | | | AATGGGTGGGCCATTTGATTG | |
| | | | TAGAGGGTCCACCACAGAAT | |
| | | | TATGGGATGCCTTAAGTG | |
| CD84 | NM_001184879.1 | 29-128 | TCTGCTAGAACAGTGCCGTGC | 12 |
| | | | TTTTCCACAGAAGGTTAGACC | |
| | | | CTGAAAGAGATGGCTCAGCA | |
| | | | CCACCTATGGATCTTGCTCCT | |
| | | | TTGCCTGCAAACCTGGC | |
| CD8A | NM_001768.5 | 1321- | GCTCAGGGCTCTTTCCTCCAC | 13 |
| | | 1420 | ACCATTCAGGTCTTTCTTTCC | |
| | | | GAGGCCCCTGTCTCAGGGTGA | |
| | | | GGTGCTTGAGTCTCCAACGGC | |
| | | | AAGGGAACAAGTACTT | |
| CDKN2A | NM_000077.3 | 976-1075 | AAGCGCACATTCATGTGGGC | 14 |
| | | | ATTTCTTGCGAGCCTCGCAGC | |
| | | | CTCCGGAAGCTGTCGACTTCA | |
| | | | TGACAAGCATTTTGTGAACTA | |
| | | | GGGAAGCTCAGGGGGGT | |
| CKB | NM_001823.4 | 321-420 | TGGGCTGCGTGGCGGGCGAC | 15 |
| | | | GAGGAGTCCTACGAAGTGTTC | |
| | | | AAGGATCTCTTCGACCCCATC | |
| | | | ATCGAGGACCGGCACGGCGG | |
| | | | CTACAAGCCCAGCGATGA | |
| COL4A3 | NM_000091.4 | 1085- | CTGGAAGTGAGGGAGTCAAG | 16 |
| | | 1184 | GGCAACAGGGGTTTCCCTGG | |
| | | | GTTAATGGGTGAAGATGGCA | |
| | | | TTAAGGGACAGAAAGGGGAC | |
| | | | ATTGGCCCTCCAGGATTTCG | |
| COL4A4 | NM_000092.4 | 761-860 | TATATGGGAGTGGAAAGAAA | 17 |
| | | | TACATTGGTCCTTGTGGAGGA | |
| | | | AGAGATTGCTCTGTTTGCCAC | |
| | | | TGTGTTCCTGAAAAGGGGTCT | |
| | | | CGGGGTCCACCAGGACC | |
| CTPS1 | NM_001301237.1 | 581-680 | ATATGATCGCTTGCTGGAGAC | 18 |
| | | | CTGCTCTATTGCCCTTGTGGG | |
| | | | CAAATACACGAAGTTCTCAG | |
| | | | ACTCCTATGCCTCTGTCATTA | |
| | | | AGGCTCTGGAGCATTCT | |
| DDX21 | NM_004728.2 | 686-785 | AACTTCTCAAAGGCCGAGGA | 19 |
| | | | GTGACCTTCCTATTTCCTATA | |
| | | | CAAGCAAAGACATTCCATCAT | |
| | | | GTTTACAGCGGGAAGGACTT | |
| | | | AATTGCACAGGCACGGAC | |
| DHX16 | NM_001164239.1 | 2491- | CCCGTGTCAACTTCTTTCTCC | 20 |
| | | 2590 | CTGGCGGTGACCACCTGGTTC | |
| | | | TGCTAAATGTTTACACACAGT | |
| | | | GGGCTGAGAGTGGTTACTCTT | |
| | | | CCCAGTGGTGCTATGA | |
| DNAJC14 | NM_032364.5 | 1167- | TCGTTTTCTTAAGCTGCTGGG | 21 |
| | | 1266 | TGCTTTGCTGCTCCTGGCTCT | |
| | | | GGCCCTCTTTTTGGGCTTTCT | |
| | | | ACAGTTGGGATGGCGGTTTCT | |
| | | | GGTGGGACTAGGTGAC | |
| EGR1 | NM_001964.2 | 1506- | GAGGCATACCAAGATCCACTT | 22 |
| | | 1605 | GCGGCAGAAGGACAAGAAAG | |
| | | | CAGACAAAAGTGTTGTGGCCT | |
| | | | CTTCGGCCACCTCCTCTCTCT | |
| | | | CTTCCTACCCGTCCCCG | |
| FAAH2 | NM_001353840.1 | 866-965 | TATATTTGGACACAAGCCTTC | 23 |
| | | | TCCAGGTGTGGTTCCCAACAA | |
| | | | AGGTCAGTTTCCCTTGGCTGT | |
| | | | GGGAGCCCAGGAGTTGTTTCT | |
| | | | GTGCACTGGTCCTATG | |
| FADS1 | NM_013402.3 | 1561- | CTTTTATCTTCTAGCCACAGT | 24 |
| | | 1660 | TCTAAGACCCAAAGTGGGGG | |
| | | | GTGGACACAGAAGTCCCTAG | |
| | | | GAGGGAAGGAGCTGTTGGGG | |
| | | | CAGGGGTGTAAATTATTTC | |
| HIVEP3 | NM_024503.4 | 184-283 | GTGTTCATGAAAAAAATGCA | 25 |
| | | | CAAAATCCTGCCTGGCCGGA | |
| | | | ATAATTCATGAAGAAGGGGC | |
| | | | TGGATCCGTGGGTCAGAGAA | |
| | | | CACAGGACCAGTTTGCCATC | |
| HMBS | NM_000190.3 | 316-415 | CATTGCTATGTCCACCACAGG | 26 |
| | | | GGACAAGATTCTTGATACTGC | |
| | | | ACTCTCTAAGATTGGAGAGA | |
| | | | AAAGCCTGTTTACCAAGGAG | |
| | | | CTTGAACATGCCCTGGAG | |
| HR | NM_005144.4 | 4561- | GGAAACTTGGGAATCATTCTG | 27 |
| | | 4660 | GCTTAAACAACACCTCCTCCT | |
| | | | GCTGCTCACTCCCGCTGAGCC | |
| | | | CACTCTACTGCCCCAGCTCCG | |
| | | | TTTCTACCACCGCATC | |
| INPP5J | NM_001002837.1 | 715-814 | GGATCATGACCTCGTGTTCTG | 28 |
| | | | GTTCGGGGACCTGAACTTCCG | |
| | | | CATTGAGAGCTATGACCTGCA | |
| | | | CTTTGTCAAGTTTGCCATCGA | |
| | | | CAGTGACCAGCTCCAT | |
| IQSEC3 | NM_015232.1 | 2177- | CAGAGGGAGGTGTTCCTCTTC | 29 |
| | | 2276 | AATGACCTGCTGGTGATTCTC | |
| | | | AAACTTTGCCCGAAGAAGAA | |
| | | | GAGCTCCTCCACGTACACCTT | |
| | | | TTGCAAGTCAGTTGGCC | |
| HN1 | NM_016185.2 | 1391- | TGGAACTAAAAGCAGTGAAG | 30 |
| | | 1490 | CAAGGGAGGCAATCACACTG | |
| | | | AAGCGGGTCTTCCTCCAGGAA | |
| | | | CGGGGTCCCACAGGCGTGTTG | |
| | | | TTTTAAATAACCTGATGC | |
| KBTBD6 | NM_152903.4 | 1371- | CATACTCGGGGGACCTTTACA | 31 |
| | | 1470 | AAGTGCCGTCACCTTTGACCT | |
| | | | GTCTGGCTCACACTAGGACTG | |
| | | | TCACCACTCTAGCTGTCTGTA | |
| | | | TCTCTCCTGACCATGA | |
| LCN8 | NM_178469.3 | 455-554 | TTCCTCACCTTGAGCGGGAGT | 32 |
| | | | AACCTGACCGTGAAGGTTGC | |
| | | | ATATAACAGCTCAGGAAGCT | |
| | | | GTGAGATAGAGAAGATCGTG | |
| | | | GGCTCAGAAATAGACAGTA | |
| LDLRAD4 | NM_001003674.2 | 3626- | TGATGATTCCATGACTCCCAC | 33 |
| | | 3725 | CTATGCAGCCTTAAAGCCAAA | |
| | | | TCCGCGTGTGTGTGTTTGTGT | |
| | | | CTGTCTGTGGGTCTCGAAGGT | |
| | | | GATCCGTCGGTGCGGT | |
| LRP8 | NM_033300.2 | 1591- | ATCTCCTCCACTGACTTCCTG | 34 |
| | | 1690 | AGCCACCCTTTTGGGATAGCT | |
| | | | GTGTTTGAGGACAAGGTGTTC | |
| | | | TGGACAGACCTGGAGAACGA | |
| | | | GGCCATTTTCAGTGCAA | |
| MFHAS1 | NM_004225.2 | 3256- | ACAGTGTCCAGATCAACAGC | 35 |
| | | 3355 | CATGTGGTGCACAGGTCGGAT | |
| | | | GGTAAATTTCAGATCTTTGCC | |
| | | | TATAGAGGGAAAGTTCCTGTG | |
| | | | GTTGTGAGTTACAGACC | |
| MTAP | NM_002451.3 | 401-500 | CTCCTTGAGGGAGGAGATTCA | 36 |
| | | | GCCCGGCGATATTGTCATTAT | |
| | | | TGATCAGTTCATTGACAGGAC | |
| | | | CACTATGAGACCTCAGTCCTT | |
| | | | CTATGATGGAAGTCAT | |
| MTHFD2 | NM_006636.3 | 1006- | TGGAGGTGTTGGCCCCATGAC | 37 |
| | | 1105 | AGTGGCAATGCTAATGAAGA | |
| | | | ATACCATTATTGCTGCAAAAA | |
| | | | AGGTGCTGAGGCTTGAAGAG | |
| | | | CGAGAAGTGCTGAAGTCT | |
| MYO10 | NM_012334.2 | 2801- | CATTCCTTCTGAGGAGGAGAT | 38 |
| | | 2900 | TTTTGCACCTGAAAAAGGCAG | |
| | | | CCATAGTTTTCCAGAAGCAAC | |
| | | | TCAGAGGTCAGATTGCTCGGA | |
| | | | GAGTTTACAGACAATT | |
| NEB | NM_004543.3 | 12896- | TGTTCATGCCCATCACTGCAA | 39 |
| | | 12995 | TGACGTTCAGAGTGAGCTGA | |
| | | | AATACAAAGCTGAACATGTG | |
| | | | AAGCAAAAAGGTCATTATGTT | |
| | | | GGTGTCCCGACGATGAGA | |
| NME1 | NM_000269.2 | 501-600 | CTTGTGGTTTCACCCTGAGGA | 40 |
| | | | ACTGGTAGATTACACGAGCTG | |
| | | | TGCTCAGAACTGGATCTATGA | |
| | | | ATGACAGGAGGGCAGACCAC | |
| | | | ATTGCTTTTCACATCCA | |
| NPM3 | NM_006993.2 | 721-820 | ACAGCCGTGGTTTTCTGATTT | 41 |
| | | | TCACCATGCCCGGGGCCTCCC | |
| | | | TTCCCACCTGCCTGTGAGAAT | |
| | | | TGGAGGTTAGTGCCTGAAGCT | |
| | | | CAGAGCTACACATTTT | |
| PLCXD2 | NM_001185106.1 | 1851- | GTTTTATTTAGTCCAGATGCT | 42 |
| | | 1950 | GAAGGATGGGAAGACCCAAG | |
| | | | CAGCTGGTGTGGAGGTCTTTC | |
| | | | CCAGTGACTCATCAGTCATTC | |
| | | | AGAAATCAAAGCCTGCA | |
| PRAG1 | XM_011543813.1 | 772-871 | GTGCACAAAGAAAAACCCTC | 43 |
| | | | ATTTCCTTACCAAGACCGGCC | |
| | | | CTCCACCCAGGAGAGCTTCCG | |
| | | | CCAGAAACTGGCTGCCTTTGC | |
| | | | TGGGACCACATCTGGCT | |
| PSD3 | NM_015310.3 | 11148- | GCATTAGCAGTTTTGGGTAAG | 44 |
| | | 11247 | CTGGCGGTACTATAACACGTA | |
| | | | CTGGAAACCTGTTCCTCATCA | |
| | | | CCACCTACCAGATTCTGGAAA | |
| | | | TGCCGTCTTCTAGAAA | |
| PTPRN2 | NM_001308267.1 | 303-402 | CGTGGCAGGATGACTATACTC | 45 |
| | | | AGTATGTGATGGACCAGGAA | |
| | | | CTTGCAGACCTCCCGAAAACC | |
| | | | TACCTGAGGCGTCCTGAAGCA | |
| | | | TCCAGCCCAGCCAGGCC | |
| PYCR1 | NM_006907.2 | 514-613 | TGGATGAAATAGGCGCCGAC | 46 |
| | | | ATTGAGGACAGACACATTGT | |
| | | | GGTGTCCTGCGCGGCCGGCGT | |
| | | | CACCATCAGCTCCATTGAGAA | |
| | | | GAAGCTGTCAGCGTTTCG | |
| RBPMS | NM_001008710.1 | 843-942 | AAACAGCCTGTAGGTTTTGTC | 47 |
| | | | AGTTTTGACAGTCGCTCAGAA | |
| | | | GCAGAGGCTGCAAAGAATGC | |
| | | | TTTGAATGGCATCCGCTTCGA | |
| | | | TCCTGAAATTCCGCAAA | |
| RCC1 | NM_001269.4 | 1885- | ATATTTGGCTCAGAACAGGTG | 48 |
| | | 1984 | TCCATGGGACAAAAAAGAAC | |
| | | | GATCCTCCACTTGACCAAGAA | |
| | | | AAAAGTGATTCTCCCAGAAG | |
| | | | CACAAAGCATACTCTTGC | |
| RNGTT | NM_003800.3 | 636-735 | CCTTTTTGGTGGAGAAAATGG | 49 |
| | | | ATTGGAGTATCGAAGCAGCA | |
| | | | GTTGCTACTTTTGCCCAAGCC | |
| | | | AGACCACCAGGAATCTACAA | |
| | | | GGGTGATTATTTGAAGGA | |
| SCIMP | NM_207103.2 | 203-302 | CCCTGAAACACAAGCAAGTA | 50 |
| | | | GATGAAGAAAAGATGTATGA | |
| | | | GAATGTTCTTAATGAGTCGCC | |
| | | | AGTTCAATTACCGCCTCTGCC | |
| | | | ACCGAGGAATTGGCCTTC | |
| FAM159A | NM_001042693.2 | 334-433 | ATTGGCGCTCTCATAGGCCTG | 51 |
| | | | TCCGTAGCAGCAGTGGTTCTT | |
| | | | CTCGCCTTCATTGTTACCGCC | |
| | | | TGTGTGCTCTGCTACCTGTTC | |
| | | | ATCAGCTCTAAGCCCC | |
| SLC16A1 | NM_003051.3 | 636-735 | TGGTGGCTGCTTGTCAGGCTG | 52 |
| | | | TGGCTTGATTGCAGCTTCTTT | |
| | | | CTGTAACACCGTACAGCAACT | |
| | | | ATACGTCTGTATTGGAGTCAT | |
| | | | TGGAGGTCTTGGGCTT | |
| SLC1A5 | NM_001145144.1 | 181-280 | GGCTTGGTAGTGTTTGCCATC | 53 |
| | | | GTCTTTGGTGTGGCGCTGCGG | |
| | | | AAGCTGGGGCCTGAAGGGGA | |
| | | | GCTGCTTATCCGCTTCTTCAA | |
| | | | CTCCTTCAATGAGGCCA | |
| SLC25A19 | NM_001126121.1 | 1086- | AGCTTGCTGAAGGCTGCCCTC | 54 |
| | | 1185 | TCCACAGGCTTCATGTTCTTC | |
| | | | TCGTATGAATTCTTCTGTAAT | |
| | | | GTCTTCCACTGCATGAACAGG | |
| | | | ACAGCCAGCCAGCGCT | |
| SLC26A8 | NM_052961.3 | 3142- | TAGCAGTACTTCCTTCCTGAC | 55 |
| | | 3241 | TGTGACTCCTACTACCTGCCA | |
| | | | GCCTTCTTCCTTGCTCTGCGCT | |
| | | | GGGATCATACTCCCAAATCAC | |
| | | | ATTACTAAATGCCAA | |
| SPATA18 | NM_145263.2 | 249-348 | GCAGCGTGGGGCCGAGAGGA | 56 |
| | | | ATAGTGAGCGATGGCGGAAA | |
| | | | ACCTGAAAAGACTGGTCTCA | |
| | | | AACGAAACTTTACGAACGTTG | |
| | | | CAGGAAAAGCTAGACTTCT | |
| SQLE | NM_003129.3 | 2649- | TAAGCTTAAAGGGGAACCAT | 57 |
| | | 2748 | TTGTGAATGAATATTTGGAAC | |
| | | | TTACCAAGTCCTAAGAGACTT | |
| | | | TTGGAAGAGGATATATATAG | |
| | | | CATAGTACCATACCACTT | |
| SSH3 | NM_017857.3 | 1235- | AACAGGGTCACCCACATCTTG | 58 |
| | | 1334 | AACATGGCCCGGGAGATTGA | |
| | | | CAACTTCTACCCTGAGCGCTT | |
| | | | CACCTACCACAATGTGCGCCT | |
| | | | CTGGGATGAGGAGTCGG | |
| TFRC | NM_003234.1 | 1221- | CAGTTTCCACCATCTCGGTCA | 59 |
| | | 1320 | TCAGGATTGCCTAATATACCT | |
| | | | GTCCAGACAATCTCCAGAGCT | |
| | | | GCTGCAGAAAAGCTGTTTGG | |
| | | | GAATATGGAAGGAGACT | |
| TMPRSS3 | NM_024022.2 | 515-614 | ACCGCTGTGTCCGGGTGGGTG | 60 |
| | | | GTCAGAATGCCGTGCTCCAGG | |
| | | | TGTTCACAGCTGCTTCGTGGA | |
| | | | AGACCATGTGCTCCGATGACT | |
| | | | GGAAGGGTCACTACGC | |
| TOMM40 | NM_001128917.1 | 436-535 | GGGCACATTCGAGGAGTGCC | 61 |
| | | | ACCGGAAGTGCAAGGAGCTG | |
| | | | TTTCCCATTCAGATGGAGGGT | |
| | | | GTCAAGCTCACAGTCAACAA | |
| | | | AGGGTTGAGTAACCATTTT | |
| TOR4A | NM_017723.2 | 3348- | TCTGGTCTGGCCTTCGTGTTG | 62 |
| | | 3447 | GCTTCAAGGTGGTAACAGAG | |
| | | | AGAGCTGCAGAGTGGGGGTG | |
| | | | TGTTTGCCAGTCCCTGCATGG | |
| | | | GTGAATGTTGGGGTGCCC | |
| TRIP13 | NM_004237.2 | 451-550 | AAGAGACAGAAAACATAATT | 63 |
| | | | GCAGCAAATCACTGGGTTCTA | |
| | | | CCTGCAGCTGAATTCCATGGG | |
| | | | CTTTGGGACAGCTTGGTATAC | |
| | | | GATGTGGAAGTCAAATC | |
| TTLL12 | NM_015140.3 | 3156- | CTATCTTTCTTGTAGCAAAGC | 64 |
| | | 3255 | TGCACCTGATGATGCTGCCTC | |
| | | | TCCTCTCTGTGTTGTCTGGGC | |
| | | | CCTTGTTTACAAGCACGCGTT | |
| | | | ACCCTTCCTGAGGGGA | |
| TVP23C | NM_145301.2 | 100-199 | TGACGGGTCGCGTCAGTTCCG | 65 |
| | | | ACCCGGACCCGTACGCTGCTG | |
| | | | CGCTGACGTGGCTCCTGGAAG | |
| | | | CAGGGCTGGCGTAGGGCCGC | |
| | | | CATGTTGCAGCAGGATA | |
| ZKSCAN5 | NM_014569.3 | 3689- | CCCAGAAATAGACCTCTCCTG | 66 |
| | | 3788 | TAGAGTGGTGATATACAGAA | |
| | | | TGAGTTTCAGTTTGCATTGCA | |
| | | | GCTGGGATTGAAAGTAATCA | |
| | | | GTCGTGAGCAGGCAGGCA | |
| ZNF346 | NM_012279.3 | 133-232 | TGCTGGAGGGCCAGGAGCCG | 67 |
| | | | GACGGGGTGCGCTTTGACCGC | |
| | | | GAGAGGGCGCGCCGCCTGTG | |
| | | | GGAAGCCGTGTCCGGTGCCC | |
| | | | AGCCGGTGGGTAGAGAGGA | |
| ZNF395 | NM_018660.2 | 4001- | TCACACGTGCTCTGTTCTCGG | 68 |
| | | 4100 | GGTTGTTCCATTCATGCCTTC | |
| | | | TTGGAGGGTGAGGGTGGCTT | |
| | | | GGGAACCGACCCAGTGATCA | |
| | | | TGCCTACTTTCTTCTTTG | |
| ZNF433 | NM_001080411.1 | 639-738 | TGTTCAGACACATGAAAGGG | 69 |
| | | | CTCATAGTGGAAGGAAACTCT | |
| | | | ATGTTTGTGAGGAATGCGGA | |
| | | | AAAACATTTATTTCCCATTCA | |
| | | | AACCTTCAAAGACACAGG | |

### I. Aspects of the Present Embodiments

The BTK inhibitor ibrutinib is used widely to treat relapsed/refractory MCL and has moved into the frontline setting in multiple clinical trials; however, this agent yields variable therapeutic benefit among patients. The mechanisms underlying intrinsic and acquired resistance to ibrutinib need to be deciphered to further improve the clinical outcomes of MCL. The present studies unravel the essential role of metabolic reprogramming in ibrutinib resistance, producing a reliance on the OXPHOS pathway. This finding contrasts with the Warburg effect asserting that glycolysis drives tumor growth and proliferation (Vander Heiden et al., 2009). OXPHOS pathway activity and glutaminolysis appear to be essential sources of energy to support proliferation in ibrutinib-resistant MCL cells, and the blockade of those pathways results in cell death, suggesting that complex I of the ETC may be a valuable therapeutic target in ibrutinib-resistant MCL cells. In addition, ibrutinib-resistant lymphomas are sensitive to BH3-mimetics (e.g., venetoclax (ABT-199)), noncovalent BTK inhibitors (e.g., Loxo-305), and CAR-T cell therapies (e.g., those targeting CD19 on the surface of lymphoma cells; see, for example, U.S. Pat. No. 10,221,245 or WO2009/091826).

### II. Methods of Treatment

The present invention provides methods of treating a cancer patient with an agent that inhibits oxidative phosphorylation, such as an IACS-010759, a BH3-mimetic, such as venetoclax (ABT-199), a noncovalent BTK inhibitor, such as Loxo-305, or a CAR-T cell therapy, such as those targeting CD19. Such treatment may also be in combination with another therapeutic regime, such as chemotherapy or immunotherapy. Certain aspects of the present invention can be used to select a cancer patient for treatment based on a gene expression signature in the patient's cancer cells. Other aspects of the present invention provide for selecting a cancer patient for treatment based on the patient having previously failed to respond to the administration of ibrutinib or who relapsed after responding to ibrutinib.

The term “subject” or “patient” as used herein refers to any individual to which the subject methods are performed. Generally the patient is human, although as will be appreciated by those in the art, the patient may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient.

“Treatment” and “treating” refer to administration or application of a therapeutic agent to a subject or performance of a procedure or modality on a subject for the purpose of obtaining a therapeutic benefit of a disease or health-related condition. For example, a treatment may include administration chemotherapy, immunotherapy, radiotherapy, performance of surgery, or any combination thereof.

The methods described herein are useful in treating cancer. Generally, the terms “cancer” and “cancerous” refer to or describe the physiological condition in mammals that is typically characterized by unregulated cell growth. More specifically, cancers that are treated in connection with the methods provided herein include, but are not limited to, solid tumors, metastatic cancers, or non-metastatic cancers. In certain embodiments, the cancer may originate in the lung, kidney, bladder, blood, bone, bone marrow, brain, breast, colon, esophagus, duodenum, small intestine, large intestine, colon, rectum, anus, gum, head, liver, nasopharynx, neck, ovary, pancreas, prostate, skin, stomach, testis, tongue, or uterus.

The cancer may specifically be of the following histological type, though it is not limited to these: neoplasm, malignant; carcinoma; non-small cell lung cancer; renal cancer; renal cell carcinoma; clear cell renal cell carcinoma; lymphoma; blastoma; sarcoma; carcinoma, undifferentiated; meningioma; brain cancer; oropharyngeal cancer; nasopharyngeal cancer; biliary cancer; pheochromocytoma; pancreatic islet cell cancer; Li-Fraumeni tumor; thyroid cancer; parathyroid cancer; pituitary tumor; adrenal gland tumor; osteogenic sarcoma tumor; neuroendocrine tumor; breast cancer; lung cancer; head and neck cancer; prostate cancer; esophageal cancer; tracheal cancer; liver cancer; bladder cancer; stomach cancer; pancreatic cancer; ovarian cancer; uterine cancer; cervical cancer; testicular cancer; colon cancer; rectal cancer; skin cancer; giant and spindle cell carcinoma; small cell carcinoma; small cell lung cancer; papillary carcinoma; oral cancer; oropharyngeal cancer; nasopharyngeal cancer; respiratory cancer; urogenital cancer; squamous cell carcinoma; lymphoepithelial carcinoma; basal cell carcinoma; pilomatrix carcinoma; transitional cell carcinoma; papillary transitional cell carcinoma; adenocarcinoma; gastrointestinal cancer; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; lentigo maligna melanoma; acral lentiginous melanoma; nodular melanoma; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; an endocrine or neuroendocrine cancer or hematopoietic cancer; pinealoma, malignant; chordoma; central or peripheral nervous system tissue cancer; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; B-cell lymphoma; malignant lymphoma; Hodgkin's disease; Hodgkin's; low grade/follicular non-Hodgkin's lymphoma; paragranuloma; malignant lymphoma, small lymphocytic; malignant lymphoma, large cell, diffuse; malignant lymphoma, follicular; mycosis fungoides; mantle cell lymphoma; Waldenstrom's macroglobulinemia; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; chronic lymphocytic leukemia (CLL); acute lymphoblastic leukemia (ALL); Hairy cell leukemia; chronic myeloblastic leukemia; and hairy cell leukemia.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

Likewise, an effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Regarding neoplastic condition treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

For the treatment of disease, the appropriate dosage of a therapeutic composition will depend on the type of disease to be treated, as defined above, the severity and course of the disease, previous therapy, the patient's clinical history and response to the agent, and the discretion of the physician. The agent may be suitably administered to the patient at one time or over a series of treatments.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. A tissue, tumor, or cell can be contacted with one or more compositions or pharmacological formulation(s) comprising one or more of the agents or by contacting the tissue, tumor, and/or cell with two or more distinct compositions or formulations. Also, it is contemplated that such a combination therapy can be used in conjunction with radiotherapy, surgical therapy, or immunotherapy.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

An anti-cancer first treatment may be administered before, during, after, or in various combinations relative to a second anti-cancer treatment. The administrations may be in intervals ranging from concurrently to minutes to days to weeks. In embodiments where the first treatment is provided to a patient separately from the second treatment, one would generally ensure that a significant period of time did not expire between the time of each delivery, such that the two compounds would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below IACS-010759, venetoclax (ABT-199), Loxo-305, or a CAR-T cell therapy is “A” and another anti-cancer therapy is “B”:

| | | | | |
|---|---|---|---|---|
| A/B/A | B/A/B | B/B/A | A/A/B | A/B/B |
| B/A/A | A/B/B/B | B/A/B/B | B/B/B/A | B/B/A/B |
| A/A/B/B | A/B/A/B | A/B/B/A | B/B/A/A | B/A/B/A |
| B/A/A/B | A/A/A/B | B/A/A/A | A/B/A/A | A/A/B/A |

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DFMO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the invention. In the context of cancer treatment, immunotherapeutics, generally, rely on the use of immune effector cells and molecules to target and destroy cancer cells. Rituximab (Rituxan®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include CD20, carcinoembryonic antigen, tyrosinase (p9′7), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, Infection Immun., 66(11):5329-5336, 1998; Christodoulides et al., Microbiology, 144(Pt 11):3027-3037, 1998); cytokine therapy, e.g., interferons α, β, and γ, IL-1, GM-CSF, and TNF (Bukowski et al., Clinical Cancer Res., 4(10):2337-2347, 1998; Davidson et al., J. Immunother., 21(5):389-398, 1998; Hellstrand et al., Acta Oncologica, 37(4):347-353, 1998); gene therapy, e.g., TNF, IL-1, IL-2, and p53 (Qin et al., Proc. Natl. Acad. Sci. USA, 95(24):14411-14416, 1998; Austin-Ward and Villaseca, Revista Medica de Chile, 126(7):838-845, 1998; U.S. Pat. Nos. 5,830,880 and 5,846,945); and monoclonal antibodies, e.g., anti-CD20, anti-ganglioside GM2, and anti-p185 (Hanibuchi et al., Int. J. Cancer, 78(4):480-485, 1998; U.S. Pat. No. 5,824,311). It is contemplated that one or more anti-cancer therapies may be employed with the antibody therapies described herein.

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering. Isolation and transfer of tumor specific T cells has been shown to be successful in treating melanoma. Novel specificities in T cells have been successfully generated through the genetic transfer of transgenic T cell receptors or chimeric antigen receptors (CARs). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors.

In one embodiment, the present application provides for a combination therapy for the treatment of cancer wherein the combination therapy comprises adoptive T cell therapy and a checkpoint inhibitor. In one aspect, the adoptive T cell therapy comprises autologous and/or allogenic T-cells. In another aspect, the autologous and/or allogenic T-cells are targeted against tumor antigens.

Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), programmed death-ligand 1 (PD-L1), T-cell immunoglobulin domain and mucin domain 3 (TIM-3), and V-domain Ig suppressor of T cell activation (VISTA). In particular, the immune checkpoint inhibitors target the PD-1 axis and/or CTLA-4.

The immune checkpoint inhibitors may be drugs, such as small molecules, recombinant forms of ligand or receptors, or antibodies, such as human antibodies (e.g., International Patent Publication WO2015/016718; Pardol, Nat Rev Cancer, 12(4): 252-264, 2012; both incorporated herein by reference). Known inhibitors of the immune checkpoint proteins or analogs thereof may be used, in particular chimerized, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95(17): 10067-10071; Camacho et al. (2004) J Clin Oncology, 22(145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present invention, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present invention to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present invention to improve the treatment efficacy.

### III. Kits and Diagnostics

Kits are envisioned containing diagnostic agents, therapeutic agents, and/or other therapeutic and delivery agents. The kit may comprise reagents capable of use in determining the expression level of at least a portion of the genes listed in Table 1. For example, reagents of the kit may include at least 20, 25, 30, 35, 40, 45, 50, 55, 60, 65, 70, 75, 80, 85, 90, 95, or 100 probes that constitute a Nanostring nCounter codeset, as well as reagents to prepare the target nucleic acids for analysis.

nCounter® systems and methods from NanoString Technologies® (as described in US2003/0013091, US2007/0166708, US2010/0015607, US2010/0261026, US2010/0262374, US2010/0112710, US2010/0047924, US2014/0371088, and US2011/0086774, each of which is incorporated herein by reference in its entirety) are a preferred means for identifying target proteins and/or target nucleic acids.

The kit may also comprise a suitable container means, which is a container that will not react with components of the kit, such as an eppendorf tube, a syringe, a bottle, or a tube. The container may be made from sterilizable materials such as plastic or glass.

The kit may further include an instruction sheet that outlines the procedural steps of the methods, such as the same procedures as described herein or are otherwise known to those of ordinary skill.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials & Methods

Sample Collection, Processing, and DNA/RNA Extraction.

Fresh specimens of surgical biopsy, bone marrow aspirates, and peripheral blood were obtained from patients with relapsed MCL after obtaining informed consent and approval by the Institutional Review Board at The University of Texas MD Anderson Cancer Center. Mononuclear cells were separated by Ficoll-Hypaque density centrifugation. Cells were isolated using anti-CD19 magnetic microbeads (Miltenyi Biotec, Auburn, Calif., USA). The freshly isolated tumor cells were maintained in RPMI-1640 (Life Technologies, Grand Island, N.Y., USA) supplemented with 10% heat-inactivated fetal bovine serum, penicillin (10,000 units/mL, Sigma, St. Louis, Mo., USA), streptomycin (10 mg/mL, Sigma), gentamicin (50 mg/mL, Sigma), and L-glutamine (29.2 mg/mL, Life Technologies). For DNA and RNA sequencing, fresh specimens were immediately placed into RNALater solution after surgical biopsy or Ficoll-Hypaque density centrifugation and selective CD19 magnetic isolation of CD19+ cells from bone marrow aspiration or peripheral blood. All procedures were performed in cold buffer or on ice. DNA and RNA extractions were performed at the MD Anderson Core Facility following standard protocols.

Whole Exome Sequencing.

Briefly, indexed libraries were prepared from 500 ng of Biorupter Ultrasonicator (Diagenode, Denville, N.J., USA)-sheared, genomic DNA using the KAPA Hyper Library Preparation Kit (KAPABiosystems, Wilmington, Mass., USA). The indexed libraries were prepared for capture with 6 cycles of preligation-mediated PCR amplification. Following amplification and reaction cleanup, the libraries were quantified fluorometrically using the Qubit™ dsDNA HS Assay (ThermoFisher, Waltham, Mass., USA) and assessed for size distribution using the Fragment Analyzer (Advanced Analytical, Ames, Iowa, USA). Library concentrations were normalized, and the libraries were multiplexed 6 libraries/pool. Each multiplexed library pool was hybridized to a probe pool from the SeqCap EZ Human Exome Enrichment Kit v3.0 (Roche-NimbleGen, Madison, Wis., USA). The enriched libraries were amplified with 8 cycles of post-capture PCR, then assessed for exon target enrichment by qPCR. The exon-enriched libraries were then assessed for size distribution using the Fragment Analyzer (Advanced Analytical) and quantified by qPCR using the KAPA Library Quantification Kit (KAPABiosystems). Sequencing was performed on the HiSeq4000 Sequencer (Illumina, San Diego, Calif., USA.), one capture (6 samples) per lane using the 76 bp paired-end configuration.

WES Data Processing and Genotyping Quality Check.

Raw output of the Illumina exome sequencing data was processed using Illumina's Consensus Assessment of Sequence And Variation (CASAVA) tool (v1.8.2) (available at support.illumina.com/sequencing/sequencing software/casava.html) for demultiplexing and conversion to FASTQ format. The FASTQ files were aligned to the human reference genome (hg19) using BWA (v0.7.5) (Li & Durbin, 2009) with 3 mismatches (2 mismatches must be in the first 40 seed regions) for a 76-base sequencing run. The aligned BAM files were then subjected to mark duplication, realignment and base recalibration using Picard (v1.112) and GATK (v3.1-1) software tools (DePristo et al., 2011). The generated BAM files were then used for downstream analysis. Genotyping quality check was performed to rule out any possible sample swapping or contamination. Briefly, germline SNPs were called using Platypus (v0.8.1) (Rimmer et al., 2014). Samples from the same patient were confirmed/identified by the percentage of genotyping-identity between them, which was defined by the fraction of identical germline alleles among the overlapping SNPs between the two samples. All samples in this study passed quality check, and no sample swapping or contamination was detected.

Somatic mutation calling, filtering, functional annotation, and expression of mutant alleles. MuTect (v1.1.4) (Cibulskis et al., 2013) was applied to identify somatic point mutations, and Pindel (v0.2.4) (Ye et al., 2009) was applied to identify small insertion and deletions (Indels). The MuTect and Pindel outputs were then run through the pipeline for filtering and annotation. Briefly, only MuTect calls marked as “KEEP” were selected and taken into the next step. For both substitutions and Indels, mutations with a low variant allelic fraction (VAF <0.02) or had a low total read coverage (<20 reads for tumor samples; <10 reads for germline sample), were removed. In addition, Indels that had an immediate repeat region within 25 base pairs downstream towards it 3′ region were also removed. After that, common variants reported by ExAc (the Exome Aggregation Consortium, available at exac.broadinstitute.org), Phase-3 1000 Genome Project (available at phase3browser.1000genomes.org/Homo_sapiens/Info/Index), or the NHLBI GO Exome Sequencing Project (ESP6500, available at evs.gs.washington.edu/EVS/) with the minor allele frequency greater than 0.5% were removed. The intronic mutations, mutations at 3′ or 5′ UTR or UTR flanking regions, silent mutations, in-frame small insertions and deletions were also removed.

To evaluate the probability of a missense mutation being functionally deleterious, dbNSFP (v3.0) (Liu et al., 2016) was applied to add prediction scores for all missense mutations from twelve commonly used functional prediction algorithms: Polyphen-2 (Adzhubei et al., 2013), SIFT (Kumar et al., 2009), MutationTaster (Schwarz et al., 2014), Mutation Assessor (Reva et al., 2011), LRT (Chun & Fay, 2009), FATHMM-MKL (Shihab et al., 2015), DANN (Quang et al., 2015), PROVEAN (Choi et al., 2012), WEST3 (Carter et al., 2013), CADD (Kircher et al., 2014), GERP++(Davydoy et al., 2010), MetaSVM, and MetaLR (Dong et al., 2015). A missense mutation that was called as “deleterious” or “damaging” by five or more algorithms were defined as a “deleterious” mutation.

DNA Copy Number Analysis.

DNA copy number analysis was conducted using an in-house application ExomeLyzer (Zhang et al., 2014) followed by CBS segmentation (Olshen et al., 2004). The segmentation files were loaded to IGV (Thorvaldsdottir et al., 2013) for visualization. R package was used to identify copy number gains (Log 2 copy ratio ≥0.5) and losses (Log 2 copy ratio ≤−0.5).

Whole Transcriptome Sequencing.

Barcoded, Illumina compatible, strand-specific total RNA libraries were prepared using the TruSeq Stranded Total RNA Sample Preparation Kit (Illumina). Briefly 250 ng of DNase I-treated total RNA was depleted of cytoplasmic and mitochondrial ribosomal RNA (rRNA) using Ribo-Zero Gold (Illumina). After purification, the RNA was fragmented using divalent cations and double-stranded cDNA was synthesized using random primers. The ends of the resulting double-stranded cDNA fragments were repaired, 5′-phosphorylated, 3′-A tailed and Illumina-specific indexed adapters were ligated. The products were purified and enriched with 12 cycles of PCR to create the final cDNA library. The libraries were quantified using the Qubit™ dsDNA HS Assay (ThermoFisher), then multiplexed into 24 libraries per pool. The pooled library was quantified by qPCR using the KAPA Library Quantification Kit (KAPABiosystems), and assessed for size distribution using the Fragment Analyzer (Advanced Analytical), then sequenced in four lanes of the Illumina HiSeq4000 sequencer using the 76 bp paired-end format.

RNA-Seq Data Processing and Quality Check.

RNA-seq FASTQ files were processed through FastQC (v0.11.5) (available at bioinformatics.babraham.ac.uk/projects/fastqc/), a quality control tool to evaluate the quality of sequencing reads at both the base and read levels and RNA-SeQC (v1.1.8) (DeLuca et al., 2012) to generate a series of quality control metrics for the RNA-seq data. One RNA sample with a low sequencing yield and poor mapping rate was excluded from this study. STAR 2-pass alignment (v2.5.3) (Dobin et al., 2013) was performed with default parameters to generate RNA-seq BAM files.

Identification of Differentially Expressed Genes and Enriched Signaling Pathways.

HTSeq-count (v0.9.1) tool was applied to the RNA-seq BAM files to count how many aligned reads overlap with the exons of each gene. The HTSeq raw count data were processed by Deseq2 (v3.6) software to identify differentially expressed genes (DEGs) between the ibrutinib-sensitive and ibrutinib-resistant phenotypes. A cut-off of fold change ≥2 or ≤−0.5 and FDR p-value ≤0.01 was applied to select the most differentially expressed genes. A ranked list of genes was generated based on DeSeq2 FDR p-values for all coding genes and processed by Gene Set Enrichment Analysis (GSEA) (Subramanian et al., 2005) against the curated gene sets from Molecular Signature Database (MSigDB) (Liberzon et al., 2015) to identify significantly enriched signaling pathways. A cut-off of FDR p-value <0.01 was applied to select the most significantly enriched signaling pathways.

nanoString Cancer Panel.

Clinical samples collected from MCL patients (n=16) were used to extract RNA, and the RNA samples were used to conduct gene expression profiling of 770 genes using nanoString® Pan Cancer Pathways Panel in the nCounter® system (Seattle, Wash., USA) at Baylor College of Medicine Core Facility, Houston, Tex., USA. Gene expression profiling analysis was adjusted for technical and biological covariates such as binding density and sample type. The analysis for multiples statistical comparisons were adjusted using the Benjamini-Yekutieli method (Benjamini & Yekutieli, 2001).

Cell Growth Assay.

Cells were seeded in triplicate in a 96-well plate with 2×10⁵ cells per well and treated with indicated dose of single agent or two compound combination for 24-72 hours. In the last 30 min, 50 μL of CellTiter 96 Aqueous One Solution Reagent were added to the culture wells and incubated at 37° C. in 5% CO₂. Light absorbance of formazan was measured at 495 nm on a universal microplate reader equipped with KC4 software (Biotek Instruments, Winooski, Vt., USA).

Apoptosis assays. Annexin V-binding assay was used to detect the induction of apoptosis. Cells were seeded in 48-well plates with single agent or combination therapy for various times, or with different concentrations for 48 hours. To quantify the percentages of cells undergoing apoptosis, Annexin V-FITC was used. Briefly, cells were washed twice with cold PBS and then suspended in binding buffer at a concentration of 1×10⁶ cells/mL. After incubation, 100 μL of the solution was transferred to a 5-mL tube, to which 5 μL of Annexin V-FITC and 5 μL of PI are added. The tube was gently vortexed and incubated for 15 min at room temperature in dark. At the end of the incubation, 300 μL of binding buffer was added. Flow cytometric analysis was performed immediately with a Novocyte Flow Cytometer (ACEA Biosciences, San Diego, Calif., USA). Data were analyzed with NovoExpress (ACEA Biosciences, San Diego, Calif., USA) or FlowJo10 (Tree Star, Ashland, Oreg., USA).

Reverse Phase Protein Array (RPPA) Assay.

PDX tumors (3 mm³) or 5×10⁶ cells were incubated with 1% SDS (with beta-mercaptoethanol) and diluted in five 2-fold serial dilutions in lysis buffer containing 1% SDS. Serial diluted lysates were arrayed on nitrocellulose-coated slides (Grace Bio-lab, Bend, Oreg., USA) with the Aushon 2470 Arrayer (Aushon BioSystems, Billerica, Mass., USA). A total of 5,808 array spots were arranged on each slide, including the spots corresponding to positive and negative controls prepared from mixed cell lysates or dilution buffer, respectively. Each slide was probed with a validated primary antibody plus a biotin-conjugated secondary antibody. Only antibodies with a Pearson correlation coefficient between RPPA and western blotting of greater than 0.7 were used. Antibodies with a single or dominant band on western blotting were assessed by direct comparison to RPPA using cell lines with differential protein expression or modulated with ligands/inhibitors or siRNA for phospho- or structural proteins, respectively. The signal obtained was amplified using a Dako Cytomation-catalyzed system (Dako, Carpinteria, Calif., USA) and visualized by DAB colorimetric reaction. The slides were scanned, analyzed, and quantified using customized Microvigene software (VigeneTech Inc, Carlisle, Mass., USA) to generate spot intensity. Each dilution curve was fitted with a logistic model (“Supercurve Fitting” developed by the Department of Bioinformatics and Computational Biology in MD Anderson Cancer Center). The fitted curve was plotted with the signal intensities—both observed and fitted—on the y-axis and the log 2-concentration of proteins on the x-axis for diagnostic purposes. The protein concentrations of each slide set were normalized by median polish, which was corrected across samples by the linear expression values using the median expression levels of all antibody experiments to calculate a loading correction factor for each sample.

Western Blot Analysis.

Cells were cultured with single agent or combination therapy in the presence or absence of caspase inhibitors. Cells were harvested, washed twice with cold PBS, and lysed with lysis buffer (Cell Signaling, Danvers, Mass., USA). Cell lysates were kept on ice for 30 min and centrifuged at 13,000 g for 10 min at 4° C. For the detection of cytosolic cytochrome c, the Cytosol Fractionation Kit (BioVision, Milpitas, Calif., USA) was utilized to acquire cytosolic fractions according to the manufacturer's instructions. Supernatants were collected, and the protein content of each fraction was determined using a Bio-Rad Bradford assay (Bio-Rad, Hercules, Calif., USA). Samples were boiled in loading buffer and separated by 10% SDS-PAGE. After electrophoresis, the proteins were transferred onto a nitrocellulose membrane (Bio-Rad), which was incubated with blocking solution (5% nonfat dry milk in PBS containing 0.05% Tween-20) for 2 h and immunoblotted with SLC16A1 (Novus, St. Louis, Mo., USA), SLC1A5, BTK, and p16INK4a, and GAPDH (Cell Signaling, Danvers, Mass., USA) antibodies. The membrane was finally visualized by incubating the membrane with a chemiluminescence Western blot kit (Pierce Biotechnology, Rockford, Ill., USA).

Histopathological analysis. Excised tissues from patients and PDX mice were fixed in 10% formalin solution, processed by standard methods, embedded in paraffin, sectioned at 5 μm, and stained with H&E. For immunohistochemical (IHC) evaluation, the tissues are stained with an antibody against human anti-CD20 (Dako, Glostrup, Denmark), counterstained with Harris hematoxylin and examined by standard light microscopy. Samples were analyzed using an Olympus BX51TF microscope equipped with UPlan FL 40×/0.75 and 20×/0.50 objective lenses (Olympus, Melville, N.Y., USA).

Pdx Model.

Six- to eight-week-old female NSG mice (Jackson Laboratory, Bar Harbor, Me., USA) were housed and monitored in an animal research facility. All experimental procedures and protocols were approved by the Institutional Animal Care and Use Committee of The University of Texas MD Anderson Cancer Center and were previously described (Zhang et al., 2017; Wang et al., 2008). Briefly, 5×10⁶ freshly isolated lymphoma cells were directly injected into human fetal bone implants within NSG-hu hosts after the mice were anesthetized with 5% isoflurane vaporizer. Mouse serum was collected, and the levels of circulating human β₂M in mouse serum (human β₂-microglobulin (β₂M) ELISA kit (Abnova Corporation, Walnut, Calif., USA)) were used to monitor tumor engraftment and burden. Once tumor growth was detected in the first generation, tumor masses were tested for human anti-CD20 expression and then passaged to next generation. After stable tumor passage, the tumor mass equally grew into next-generation mice (5 mice/group) for in vivo treatment. Five days after tumor implantation, the mice were administered vehicle control, ibrutinib (50 mg/kg, oral gavage daily), or IACS-010759 (10 mg/kg, oral gavage for 5 consecutive days per week) until the endpoint (one diameter of tumor mass reached 15 mm). During and after treatment with vehicle control or the indicated agent, mouse serum was collected, and tumor burden was evaluated by measuring either human β₂M levels or tumor volume with a caliper to determine therapeutic efficacy in the PDX models. Freshly isolated cells from the tumor mass of each generation and treatment group were labeled with FITC/PE-conjugated anti-human CD5, CD19, and CD20 mAb (BD Biosciences, San Diego, Calif., USA) to validate the population of human lymphoma cells by flow cytometry.

Mitochondria membrane potential, mitochondria mass, ROS, and apoptosis analysis. MCL cells were seeded and cultured overnight before treatment to reach log phase. Respective cell lines were plated at 2×10⁶ cells/well in 6-well plates in phenol-red and pyruvate-free DMEM assay medium containing 5 mM glucose (Sigma), 2 mM GlutaMAX (Gibco, ThermoFisher Scientific), and 5% dialyzed FCS (Gibco). Vehicle, IACS-0107059 (20 nM), or (1-5 μM) BPTES was added and replenished every day until the indicated time points. ROS and mitochondria membrane potential were evaluated 24 hours after treatment. To determine mitochondria membrane potential (Δψm), treated cells were loaded with MitoStatus tetramethylrhodamine, ethyl ester (TMRE) (BD Biosciences) at 37° C. for 15 min and washed with PBS twice to remove residual TMRE. TMRE dye fluorescence retained in the cells was detected by flow cytometry with excitation and emission of 548/574 nm. Data were analyzed with NovoExpress (ACEA Biosciences, San Diego, Calif., USA) or FlowJo10 (Tree Star, Ashland, Oreg., USA).

To measure ROS levels, treated cells were harvested, stained with 0.5 mM CM-H2DCF-DA (C6827, Molecular Probes, Invitrogen) in PBS for 30 min at 37° C., washed twice with PBS, and analyzed in a flow cytometer (FACS Caliber, BD Biosciences) by recording the green fluorescence of oxidized DCF. The mean fluorescence of 10,000 analyzed cells (corrected for auto fluorescence) was taken as a readout for the intracellular ROS load.

Mitochondria Respiration.

The oxygen consumption rate (OCR) was measured using a Seahorse XF-96 Metabolic Flux Analyzer (Seahorse Bioscience, North Billerica, Mass., USA). Briefly, MCL cells were treated with/without IACS-010759 (20 nM) for 1 h. On the day of analysis, 2-4×10⁵ MCL cells/well were adhered to XF 96-well cell culture microplates (Seahorse Biosciences) pre-coated with Cell-Talc (BD Biosciences), by centrifugation at 1,000×g, without brakes for 5 min, then changed to mitochondria stress medium supplemented with 25 mM glucose, 2 mM sodium pyruvate, and 2 mM glutamine (pH 7.4), followed by incubation at 37° C. in a non-CO₂ incubator for 1 h. Oligomycin (μM), FCCP (2 μM), and Rotenone/Antimycin A (0.5 μM), were loaded into drug delivery ports and injected sequentially at the indicated time points. After each injection, 4 time points were recorded with approximately 35 min between each injection. ATP consumption over time was normalized to protein content measured by sulforhodamine B (SRB) staining and plotted using Graph-Pad Prism.

Targeted Metabolomics Analysis.

MCL cell lines were grown in complete RPMI-1640 media for 2 days. Next, 1×10⁷ cells were seeded in fresh media in T-75 Flasks, then harvested either immediately (0 time point) or 24 hours later after cell counting, and washed with PBS. Metabolites were immediately extracted and subjected to targeted metabolomics analysis for amino acids and TCA metabolites. Electrospray ionization-mass spectrometry (ESI-MS) (cation) or Agilent 6460 TripleQuad LC/MS (anion) was conducted. Each metabolite was identified and quantified based on the peak information including m/z, migration time, and peak area. The peak area was then converted to relative peak area by normalization with internal standard peak area and total cell number. Relative levels of metabolites were plotted as a heatmap using Graph-Pad Prism.

Glucose, Glutamine Consumption, Lactate Production and ATP Analysis.

MCL cell lines were seeded at 1-2×10⁶ cells/well in triplicate in six-well plates and incubated overnight. Next, the media were replaced with 3 mL complete RPMI-1640 medium with or without inhibitors. Cell culture supernatants were collected 2 days after treatment with IACS-010759 or BPTES and analyzed for their glucose, glutamine and lactate content using the Glucose, Glutamine and Lactate Colorimetric Assay Kits (BioVision), respectively, according to the manufacturer's instructions. Glucose or glutamine consumption was extrapolated by subtracting the measured glucose concentrations in the media from the initial concentrations (11.1 mM glucose; 2 mM glutamine). Intracellular ATP contents from above treated cells was determined using ATP Colorimetric Assay Kit (Biovision) as outlined in the manufacturer's protocol. Relative levels of all metabolites were then normalized to the relative sum of total cell counts over a 48-hour treatment period.

Proteomics Analysis.

Cell cultures of parental JeKo-1 and two BTK knockdown clones (KD1 and KD2) were prepared in triplicate. Cells were harvested and pellets were lysed in buffer containing 2% SDS, 100 mM Tris-HCl pH 7.6. Proteins were reduced, alkylated, and digested using filter-aided sample preparation (FASP) based on a previous method with a minor modification (Wisniewski et al., 2011). One hundred micrograms of protein from each sample was incubated with 10% of 1 M DTT at 95° C. for 5 minutes. Protein extracts were alkylated using FASP in 50 mM iodoacetamide in 8 M urea, 100 mM Tris-HCl, pH 8.5. Proteins were then digested with sequencing grade porcine trypsin (Promega) at 1:100 enzyme to protein ratio in 50 mM triethylammonium bicarbonate (TEAB). Digested peptides were cleaned by 50 mg Sep-Pak SPE (Waters Milford, Mass.) before TMT labeling. Tryptic peptides were then labeled using two tandem mass tag 10-plex isobaric label reagent set (Thermo, Rockford, Ill.) following the manufacturer's instructions. Peptide concentration was determined by Pierce Quantitative Fluorometric Assay (Thermo, Rockford, Ill.), and all samples were normalized at peptide level before TMT labeling. To deal with the TMT batch effect, the experiment was blocked based on genotype and treatment to ensure that both TMT batches had similar amount of replicate of each group. A pooled sample was made from all 18 samples and then was aliquoted into two as the TMT10 in each batch which was later used for data normalization. A small portion of each labeled sample was pooled and used for a quick LC-MS/MS run which provided median ratios for another normalization before mixing the real samples. The combined samples of each TMT experiment were cleaned by 50 mg Sep-Pak SPE immediately before offline high pH fractionation. Labeled peptides were separated into 13 fractions on a 100×1.0 mm Acquity BEH C18 column (Waters, Milford, Mass.) using an UltiMate 3000 UHPLC system (Thermo, Rockford, Ill.) with a 40 min gradient from 99:1 to 60:40 buffer A:B ratio under basic pH conditions, and then consolidated into 13 super-fractions. Buffer A=0.1% formic acid, 0.5% acetonitrile; buffer B=0.1% formic acid, 99.9% acetonitrile. Both buffers adjusted to pH 10 with ammonium hydroxide for offline separation. Each super-fraction was then further separated by reverse phase Jupiter Proteo resin (Phenomenex, Torrance, Calif., USA) on an in-line 200×0.075 mm column using a nanoAcquity UPLC system (Waters Corporation, Milford, Mass., USA). Peptides were eluted using a 60 min gradient from 97:3 to 67:33 buffer A:B ratio. Buffer A=0.1% formic acid, 0.5% acetonitrile; buffer B=0.1% formic acid, 99.9% acetonitrile. Eluted peptides were ionized by electrospray (2.15 kV) followed by mass spectrometric analysis on an Orbitrap Fusion Tribrid mass spectrometer (Thermo, Rockford, Ill.) using multi-notch MS3 parameters. MS data were acquired using the FTMS analyzer in top-speed profile mode at a resolution of 240,000 over a range of 375 to 1500 m/z.

Following CID activation with normalized collision energy of 35.0, MS/MS data were acquired using the ion trap analyzer in centroid mode over a range of 400-2000 m/z. Using synchronous precursor selection, up to 10 MS/MS precursors were selected for HCD activation with normalized collision energy of 65.0, followed by acquisition of MS3 reporter ion data using the FTMS analyzer in profile mode at a resolution of 30,000 over a range of 100-500 m/z.

Proteins were identified, and reporter ions were quantified using MaxQuant (Max Planck Institute of Biochemistry, Martinsried, Germany; version 1.5.8.3) with a parent ion tolerance of 3 ppm, a fragment ion tolerance of 0.5 Da, and a reporter ion tolerance of 0.01 Da. Protein identifications were accepted if they could be established at greater than 51.0% probability to achieve an FDR less than 1.0% and contained at least 2 identified peptides. Intra- and inter-TMT batch normalization was performed using the median reporter ion intensity of each channel and pooled sample (TMT10). TMT batch-related missing values were removed and the rest of the missing values were imputated by half minimum value of the entire data set. A total of 5,393 proteins were identified, among which 5,165 proteins were mapped in Ingenuity Pathway Analysis (IPA) version 42012434 (Ingenuity Systems, available at ingenuity.com, Mountain View, Calif.). To explore the proteomic change due to BTK knockdown, BTK KD1 and KD2 were combined and compared with parental JeKo-1. Differentially expressed proteins were determined by a two-way ANOVA model built by TMT batch and genotype (KO and WT) with multiple test correction (Benjamini-Hochberg FDR <0.05) to adjust the TMT batch effect. Among all significantly changed proteins, 478 proteins containing twofold up- or down-regulated proteins were analyzed by IPA (281 down and 197 up). Right-tailed Fisher's exact test was used to calculate the p-value and p-value <0.05 was considered significant in the canonical pathway.

Statistical Analysis.

All assays were performed in triplicate and expressed as mean±SEM or SD. Statistical significance of differences observed between experimental groups was determined by Student's t-test, linear regression model, mixed-effects regression model and beta regression models. Overall survival and progression-free survival were estimated using the Kaplan-Meier method and compared using log-rank test. All analyses were performed using statistical software R v3.4.3 with packages betareg v3.1-0, nlme v3.1-131 and survival v2.41-3 and plotted using package survminer v0.4.2 and software GraphPad Prism v7.03. P-values less than 0.05 were considered significant.

### Example 1—Landscape of Somatic Mutations and DNA Copy Number Alterations

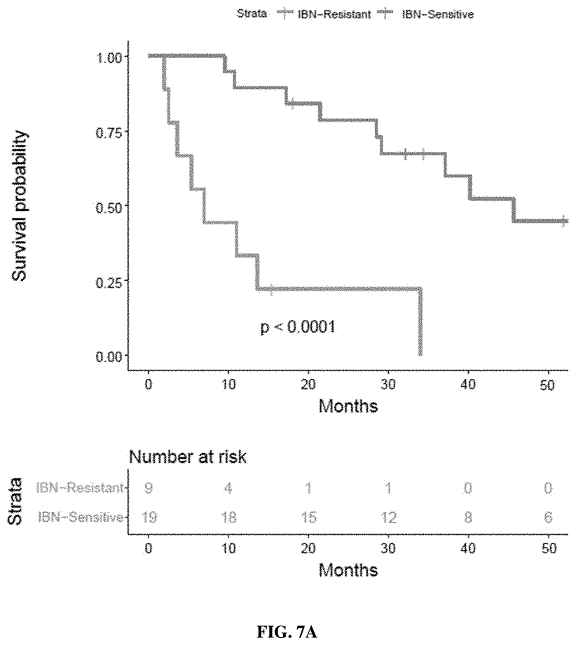

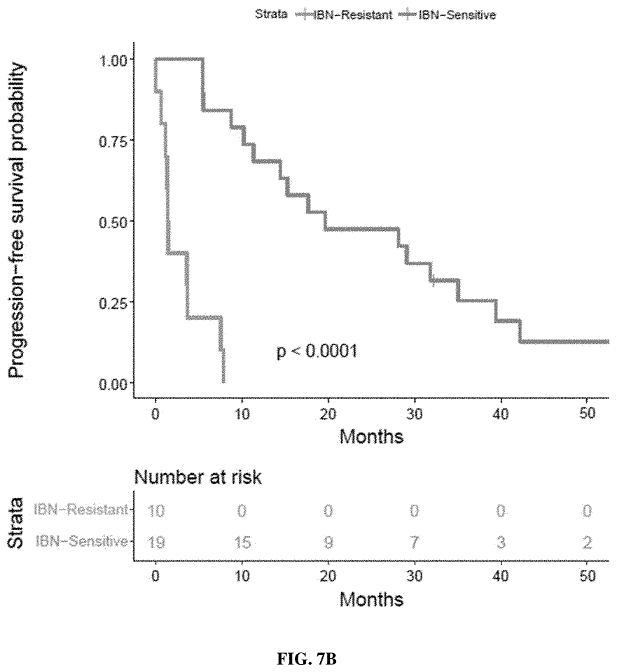

To decipher the mechanisms underlying ibrutinib resistance, clinical specimens were collected from 37 MCL patients treated with ibrutinib. The clinical and pathological information is listed in the Table 2. MCL sensitivity to ibrutinib was significantly associated with clinical outcomes, with the ibrutinib-resistant MCL patients experiencing poorer progression-free survival (PFS) and overall survival (OS) (FIG. 7).

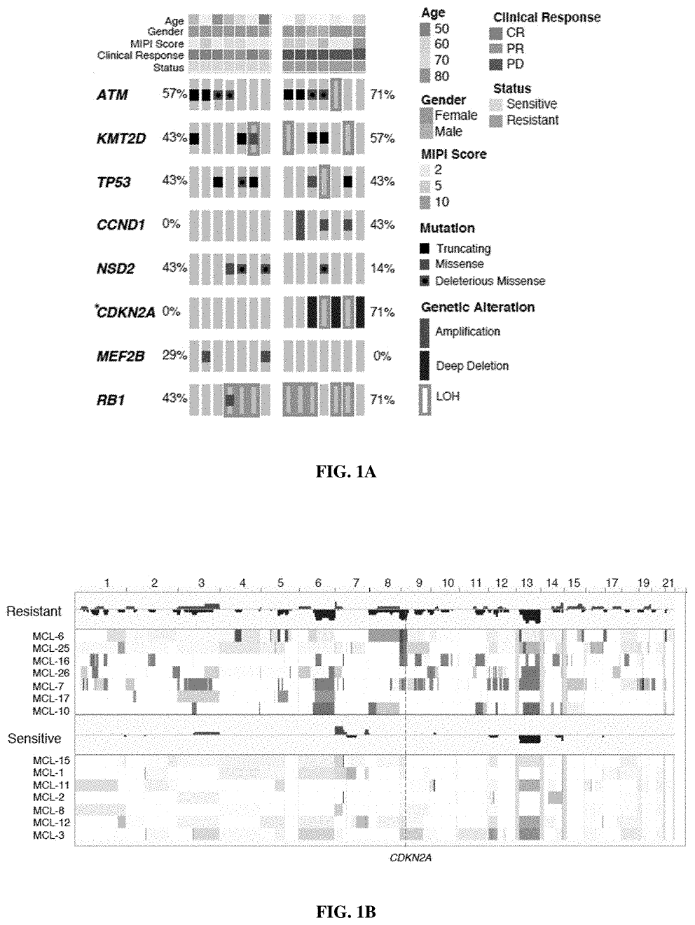

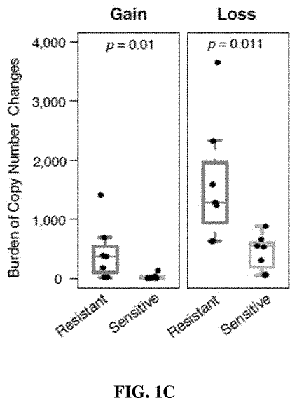

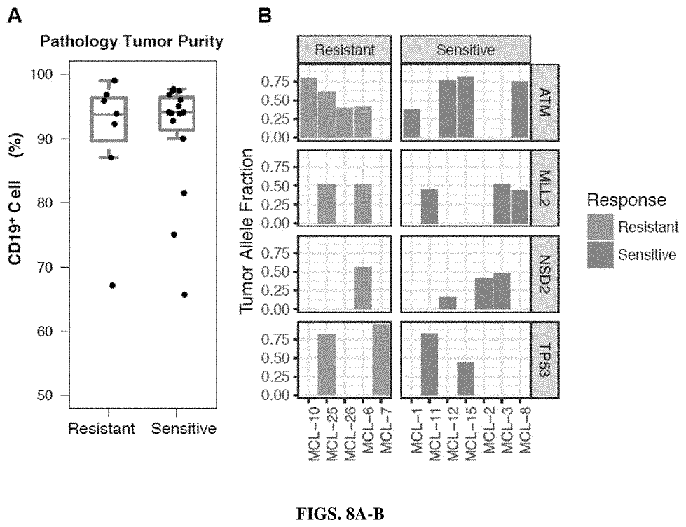

Of those 37 MCL patients, whole-exome sequencing (WES) was conducted on the clinical specimens of 14 cases that had sufficient isolated tumor DNA, including seven ibrutinib-sensitive cases and seven ibrutinib-resistant cases. The corresponding peripheral blood samples were also sequenced and used as the matched germline controls for somatic mutation and DNA copy number analysis. WES data analysis identified frequent inactivating somatic alterations in ATM, KMT2D, and TP53 in both the ibrutinib-sensitive and -resistant tumors (FIG. 1A). Of note, CDKN2A (5/7, 71%) was frequently deleted, and the deletion was only observed in the ibrutinib-resistant tumors in our cohort (p=0.010). Similarly, mutation and amplification of CCND1 were identified in only the ibrutinib-resistant tumors. DNA copy number analysis identified extensive copy number alterations, especially the copy number losses in ibrutinib-resistant tumors, including broad deletions of 6q, 9p and chromosome 13 (FIG. 1B). The burden of copy number gains and losses was dramatically increased in the ibrutinib-resistant tumors (FIG. 1C), and the increased burden of copy number changes was unlikely an artifact caused by differences in the tumor cellularity between samples in the two groups. No difference in the pathological tumor cellularity (indicated by the percentage of CD19+ cells) between the two groups was observed (FIG. 8A). In accordance, no statistical difference was observed in the distribution of tumor variant allelic fractions of all the somatic mutations identified from frequently mutated genes between the two groups (FIG. 8B). Therefore, the increased aneuploidy observed in the resistant group is most likely biologically meaningful and may have played a role in driving tumor progression during the course of treatment with ibrutinib.

TABLE 2
The clinical and pathological data of MCL patients.

| | | | | | | | | Clinical | | |
|---|---|---|---|---|---|---|---|---|---|---|
| Sample | | | MIPI | | | | | Response | Ibrutinib | Ibrutinib |
| Name | Age | Gender | Score | Ki-67 | PAX-5 | SOX11 | Prior Treatment | to ibrutinib | (days) | Status |
| MCL-1 | 59 | Male | 3 | 65% | + | N/A | R-Hyper-CVAD × 4; R-EPOCH × 4; | CR | 510 | Sensitive |
| | | | | | | | Rituximab × 4; Velcade × 4; | | | |
| | | | | | | | R-Revlimid × 1; R-Bendamustine × 1; | | | |
| | | | | | | | Temsirolimus × 1; Methotrexate and | | | |
| | | | | | | | Cytarabine × 1 | | | |
| MCL-2 | 76 | Male | 4 | N/A | N/A | N/A | R-hyper-CVAD × 6: | PR | 150 | Sensitive |
| | | | | | | | BCR (bortezomib/cytoxan.rituximab) × 6 | | | |
| MCL-3 | 50 | Male | 3 | 60% | + | − | R-hyper-CVAD alternating with | PR | 210 | Sensitive |
| | | | | | | | methotrexate and cytarabine × 6; | | | |
| | | | | | | | Bortezomib × 3; R-Bendamustine × 2; | | | |
| | | | | | | | Radiation therapy; R-lenalidomide × 12; | | | |
| | | | | | | | R-bortezomib + cytoxan × 1 | | | |
| | | | | | | | R-bortezomib × 1; | | | |
| | | | | | | | R-cytoxan + cladribine × 1 | | | |
| MCL-4 | 68 | Male | 2 | 55% | + | N/A | R-hyper-CVAD + vortezomib × 5 | PR | 150 | Sensitive |
| MCL-5 | 73 | Male | 5 | 5% | N/A | N/A | Hyper-CVAD with methotrexate and | CR | 1680 | Sensitive |
| | | | | | | | cytarabine × 6; Bortezomib × 4 | | | |
| MCL-6 | 69 | Female | 6 | 95% | N/A | N/A | Hyper-CVAD × 2; Rituximab × 8; | CR to PD | 240 | Acquired |
| | | | | | | | lenalidomide × 1; Bortezomib × 4 | | | Resistance |
| MCL-7 | 67 | Male | 3 | 90% | + | N/A | R-Hyper-CVAD with methotrexate and | CR to PD | 210 | Acquired |
| | | | | | | | cytarabine × 7; Bortezomib × 1 | | | Resistance |
| MCL-8 | 72 | Male | 5 | 20% | + | + | R-CHOP × 6; Rituximab × 8 | CR | 165 | Sensitive |
| MCL-9 | 66 | Female | 2 | 30% | + | + | R-CHOP × 4 | CR | 1710 | Sensitive |
| MCL-10 | 70 | Male | 5 | 60% | N/A | N/A | R-Hyper-CVAD with methotrexate and | PD | 120 | Primary |
| | | | | | | | cytarabine × 6 | | | Resistance |
| MCL-11 | 67 | Male | 3 | 20% | N/A | + | CHOP × 8; R-Hyper-CVAD with | CR | 1180 | Sensitive |
| | | | | | | | R + methotrexate + cytarabine × 6; | | | |
| | | | | | | | Radiation therapy; Bortezomib × 2; | | | |
| | | | | | | | JAK-2 inhibitor × 1; R-bendamustine | | | |
| | | | | | | | and bortezomib × 1 | | | |
| MCL-12 | 59 | Male | 4 | N/A | N/A | N/A | R-CHOP × 6; R-Hyper-CVAD | PR | 990 | Sensitive |
| | | | | | | | alternating with | | | |
| | | | | | | | R + methotrexate + cytarabine × 1; | | | |
| | | | | | | | Rituximab × 12; R-bortezomib × 5 | | | |
| MCL-13 | 79 | Male | 4 | 35% | + | + | R-CHOP × 1; Bortezomin × 1; | CR | 450 | Sensitive |
| | | | | | | | R-Bendamustine × 1; Lenalidomide; | | | |
| | | | | | | | R-cladribine + cytoxan × 1 | | | |
| MCL- | 88 | Male | 3 | 45% | + | N/A | R-CHOP × 6; R-bendamustine × 4; | CR | 780 | Sensitive |
| 14/15 | | | | | | | R-bortezomib and dexamethasone × 2; | | | |
| | | | | | | | Bendamustine × 1 | | | |
| MCL-16 | 70 | Male | 3 | 30% | N/A | − | R-Hyper-CVAD and bortezomib | CR to PD | 780 | Acquired |
| | | | | | | | alternating with | | | Resistance |
| | | | | | | | methotrexate + cytarabine × 6; | | | |
| | | | | | | | R-bendamustine and bortezomib × 1 | | | |
| MCL-17 | 63 | Male | 8 | N/A | N/A | N/A | R-CHOP × 4; RICE × 2; ASCT; | PD | 70 | Primary |
| | | | | | | | R-cytoxan × 1 | | | Resistance |
| MCL-18 | 62 | Male | 8 | 80% | + | N/A | R-Hyper-CVAD alternating with | PR | 10 | Sensitive |
| | | | | | | | R-methotrexate and cytarabine × 6 | | | |
| MCL-19 | 83 | Male | 11 | 65% | + | + | R-CHOP × 6; R-bortezomib × 1 | PD | 150 | Primary |
| | | | | | | | | | | Resistance |
| MCL-20 | 66 | Male | 6 | 20% | + | N/A | R-CHOP × 1; R-DHAP × 1 | PR | 485 | Sensitive |
| MCL-21 | 74 | Female | 10 | 90% | + | + | R-bendamustine × 3 | PD | 75 | Primary |
| | | | | | | | | | | Resistance |
| MCL-22 | 49 | Female | 4 | 5% | N/A | − | None | CR | 120 | Sensitive |
| MCL-23 | 68 | Male | 6 | 40% | + | N/A | R-hyper-CVAD alternating with | PD | 28 | Primary |
| | | | | | | | R-methotrexate and cytarabine × 2 | | | Resistance |
| MCL-24 | 78 | Male | 8 | 10% | + | + | R-bendamustine × 2 | PR | 20 | Sensitive |
| MCL-25 | 76 | Male | 7 | 95% | + | + | R-bendamustine × 4; Rituximab × 12; | PD | 60 | Primary |
| | | | | | | | Radiotherapy | | | Resistance |
| MCL-26 | 71 | Female | 4 | 70% | N/A | − | R-Hyper-CVAD × 5; Bortezomib × 2; | CR to PD | 430 | Acquired |
| | | | | | | | R-bendamusiine and dexamethasone × 1; | | | Resistance |
| | | | | | | | R-DHAP × 1 | | | |
| MCL-27 | 72 | Male | 7 | 95% | + | − | R-Hyper-CVAD × 4; | PD | 8 | resistant |
| | | | | | | | R-bendamustine × 4 | | | |
| MCL-28 | 44 | Male | 1 | 20% | − | − | None | CR | 196 | sensitive |
| MCL-29 | 69 | Male | 5 | ukn | − | − | None | CR to PD | 336 | Acquired |
| | | | | | | | | | | Resistance |
| MCL-30 | 61 | Female | 6 | 30% | + | − | R-Hyper-CVAD × 4; | CR | 238 | Sensitive |
| | | | | | | | R-bendamustine × 1 | | | |
| MCL-31 | 67 | Male | 5 | 80% | + | − | None | PD | 44 | Resistant |
| MCL-32 | 51 | Male | 6 | <5% | + | + | None | PR | 210 | Sensitive |
| MCL-33 | 67 | Male | 5 | 80% | + | − | None | PD | 35 | Resistant |
| MCL-34 | 70 | Male | 2 | 90%-95% | + | + | R-hyper-CVAD alternating with | CR to PD | 1000 | Acquired |
| | | | | | | | R-bortezomib and methotrexate and | | | Resistance |
| | | | | | | | cytarabine × 8 | | | |
| MCL-35 | 72 | Female | 10 | N/A | N/A | N/A | R-CHOP × 5; R-DHAP × 2 | CR | 840 | Sensitive |
| MCL-36 | 89 | Female | 5 | 30-50% | + | − | R-bendamustine × 6 | CR to PD | 1095 | Resistant |
| MCL-37 | 65 | Male | 8 | 90% | − | + | R-Hyper-CVAD and R-methotrexate and | PD | 35 | Resistant |
| | | | | | | | cytarabine × 2 | | | |

Mantle cell lymphoma international prognostic index (MIPI);
MIPI score, 0-3, low risk; 4-5, intermediate risk; 6-11, high risk
R: Rituximab;
R-Hyper-CVAD: Riluximab, cyclophosphamide, vincristine, doxorubicin, and dexamethasone;
R-CHOP: Rituxiniab, cyclophosphamide, doxorubicin, vincristine, prednisolone;
R-DHAP: Rituximab, cytarabinc. cisplatin. and dexamethasone

### Example 2—Transcriptomic Profiling Identifies Metabolic Reprogramming as a Hallmark of Ibrutinib Resistance

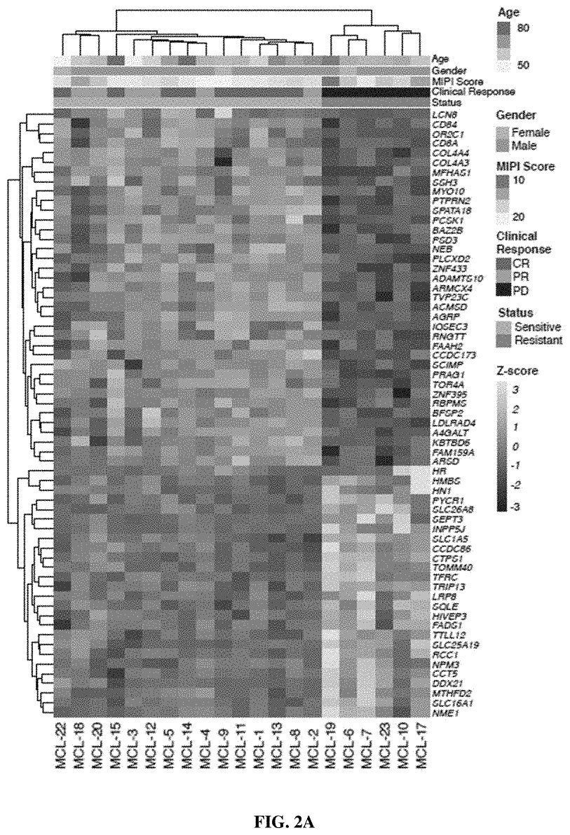

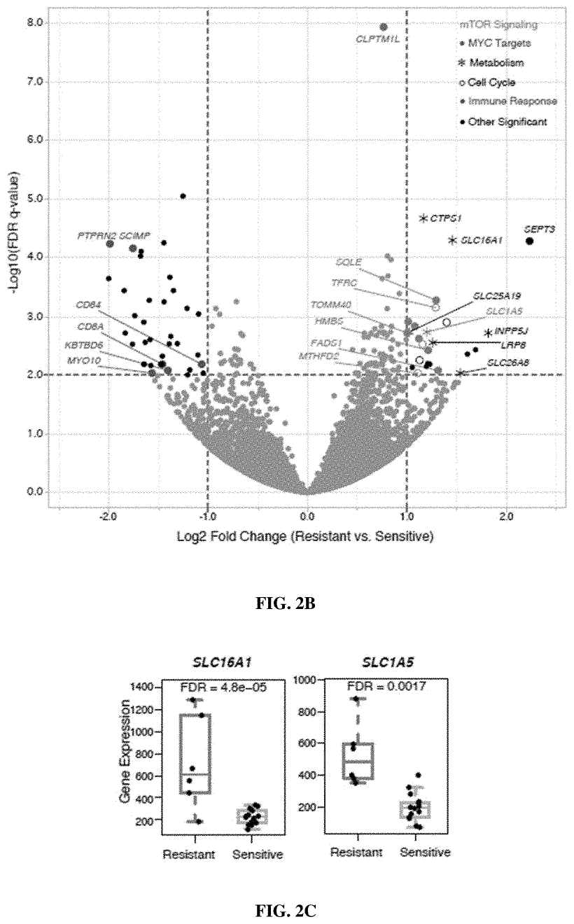

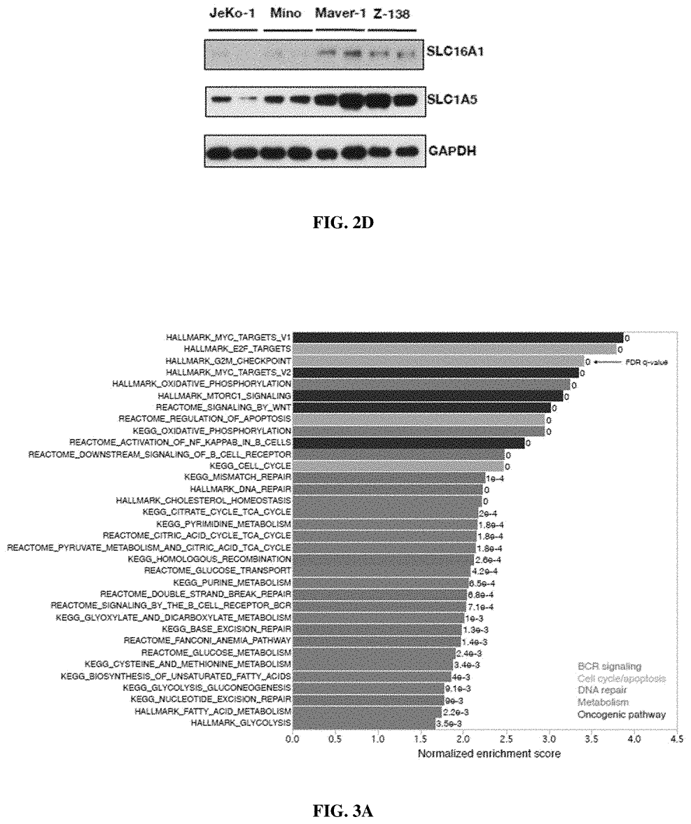

Whole transcriptome sequencing (RNA-seq) was performed on clinical specimens isolated from 14 ibrutinib-sensitive and 7 ibrutinib-resistant cases. Unsupervised hierarchical clustering of MCL tumors using RNA-seq gene expression data showed a response-specific gene expression signature (FIG. 2A). A total of 69 protein-coding genes were identified as the most differentially expressed genes (DEGs) between the ibrutinib-resistant and -sensitive groups, with a fold change of ≥2 or ≤−2 and the false discovery rate (FDR q-value)≤0.01. Among the DEGs, 26 genes were upregulated in ibrutinib-resistant tumors (FIG. 2B). Computational overlapping of those upregulated genes with the Molecular Signature Database (MSigDB, Broad Institute) hallmark gene sets suggested a significant enrichment of genes in mTOR signaling (7/26, FDR q-value=5.98e-10), cell cycle regulation (5/26, FDR q-value=1.84e-6), and MYC targets (3/26, FDR q-value=2.9e-3).

Interestingly, manual inspection of the remaining upregulated DEGs against currently available knowledge bases suggested that many of the upregulated DEGS are metabolism-related (labeled as asterisks in FIG. 2B), including SLC16A1, SLC1A5 (FIG. 2C), SLC25A19, and SLC26A. In particular, SLC25A19 is a mitochondrial transporter that mediates the uptake of thiamine pyrophosphate (TPP) and is also a cofactor for several dehydrogenase enzyme reactions, including pyruvate dehydrogenase (linking glycolysis to the TCA cycle), alpha-ketoglutarate (α-KG, linking glutamate to the TCA cycle), transketolase (HMP shunt), and branched-chain ketoacid dehydrogenase. SLC16A1 encodes a proton-coupled monocarboxylate transporter (also known as MCT-1), which is an essential transporter of lactate and pyruvate observed to be upregulated in many solid tumors (Pinheiro et al., 2012) and also maintains the metabolic phenotype of tumor cells (Baltazar et al., 2014). SLC1A5, also known as ASCT2, is a heavily studied glutamine-specific transporter regulated by c-MYC (Altman et al., 2016). Consistent with the RNA-seq analysis, ibrutinib-resistant MCL cell lines (Maver-1 and Z138) (Rahal et al., 2014) also showed increased SLC16A1 and SLC1A5 at the protein level compared with the ibrutinib-sensitive cell lines (Mino and JeKo-1) (FIG. 2D).

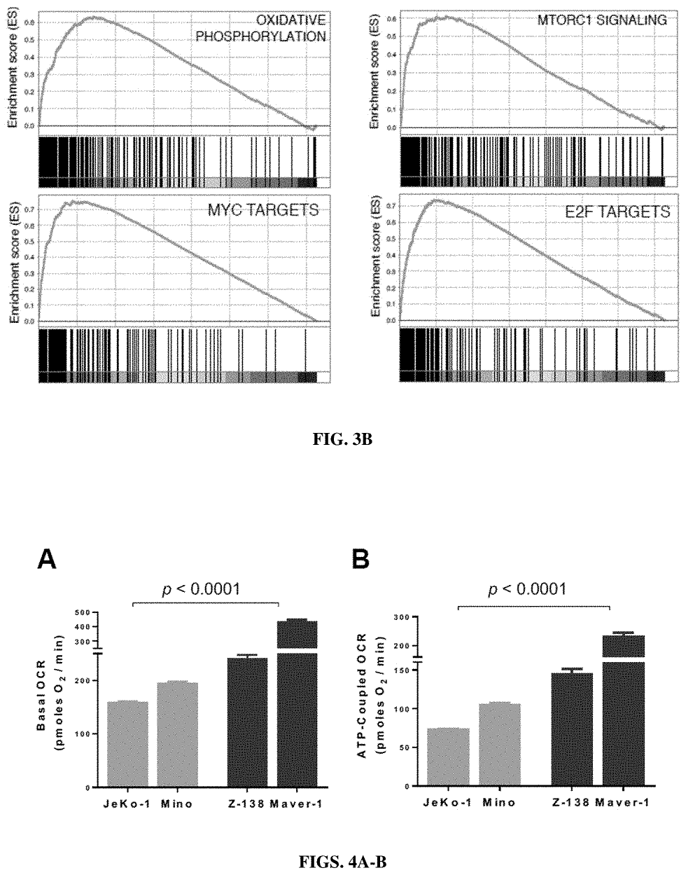

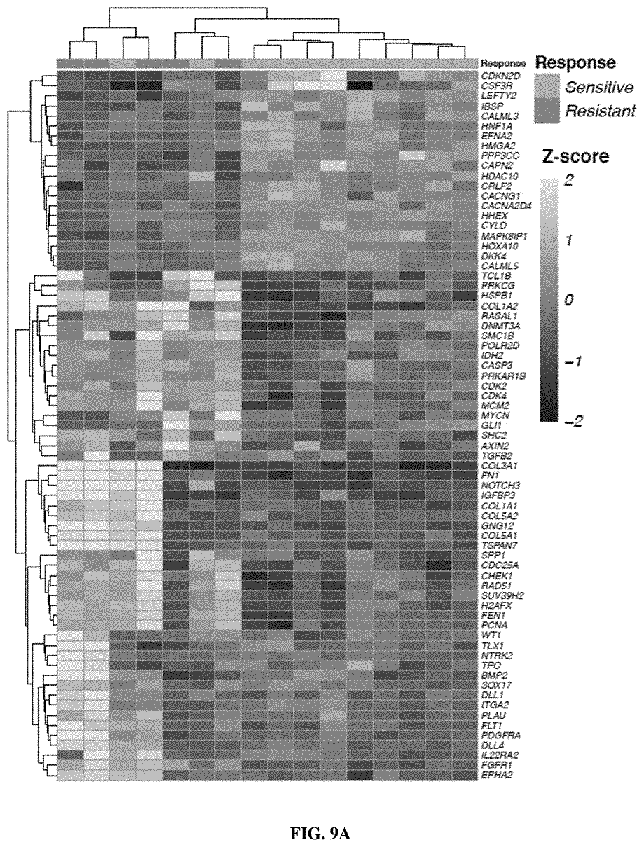

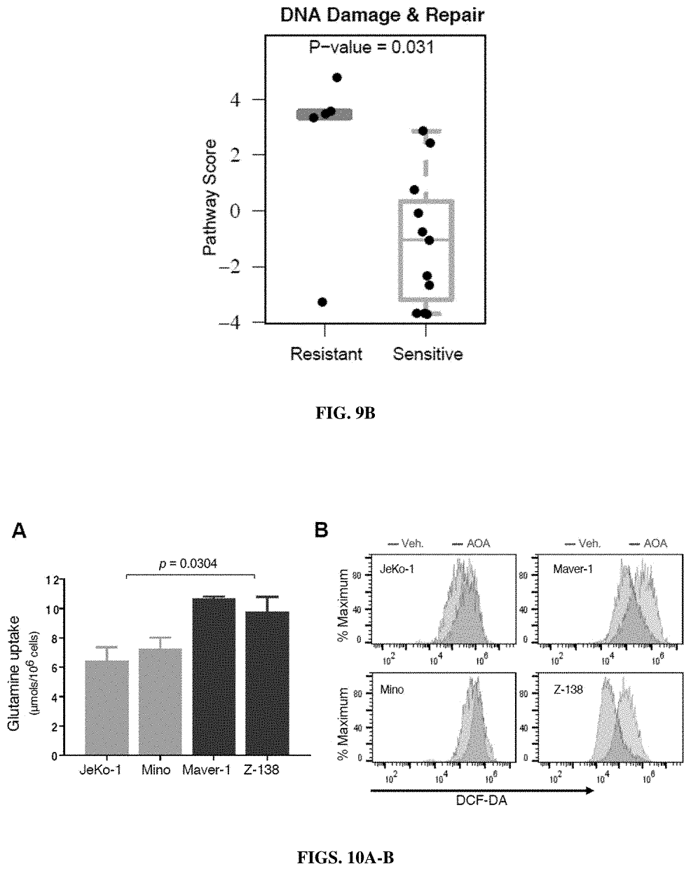

Gene set enrichment analysis (GSEA) was performed to determine whether an a priori defined set of genes showed statistically significant and concordant differences between the ibrutinib-sensitive and -resistant tumors. As shown in FIG. 3A, oncogenic pathways including c-MYC, mTOR (mTORC1), Wnt, and NF-κB signaling, followed by cell cycle, apoptosis, BCR signaling, and DNA repair, were among the most dramatically enriched pathways in the ibrutinib-resistant tumors, some of which were also confirmed by the nanoString nCounter analysis of additional clinical specimens. For example, in the nanoString nCounter analysis, SMC1B, FGFR1, and CDK2 were among the top 20 DEGs and belong to cell cycle control, MAPK, and PI3K pathways, respectively (FIG. 9A). Additionally, ibrutinib-resistant samples had significantly higher expression of PI3K and NOTCH pathway genes and lower expression of JAK-STAT pathway genes as compared with the sensitive samples. The DNA damage repair pathway was also significantly upregulated as demonstrated by both the RNA-seq and nanoString data (FIGS. 3A & 9B).

Notably, in addition to these oncogenic pathways, the metabolic pathways, including oxidative phosphorylation (OXPHOS), were significantly enriched in the ibrutinib-resistant tumors (normalized enrichment score >3 and FDR q-value <1e-5) (Zhang et al., 2019). The representative GSEA enrichment plots of OXPHOS, mTORC1 signaling, MYC targets, and E2F targets are shown in FIG. 3B. Interestingly, the upregulation of c-MYC targets and mTORC1 signaling components is consistent with the increased activity of OXPHOS because these oncogenic pathways are directly linked to metabolic reprogramming in cancer cells (Altman et al., 2016). Glutaminolysis fuels OXPHOS (DeBerardinis et al., 2007), supporting the hypothesis that ibrutinib-resistant MCL cells rely on oxidative glutamine metabolism for energy production and biosynthesis (Altman et al., 2016).

### Example 3—Ibrutinib-Resistant MCL Cells Rely on Glutamine-Fueled OXPHOS

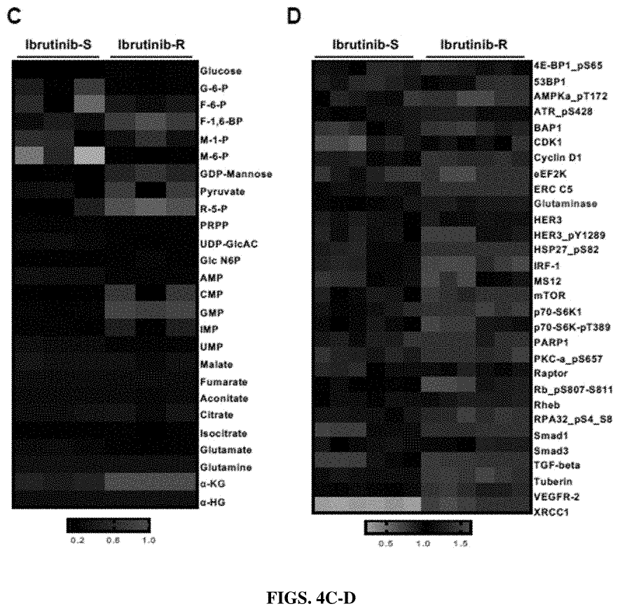

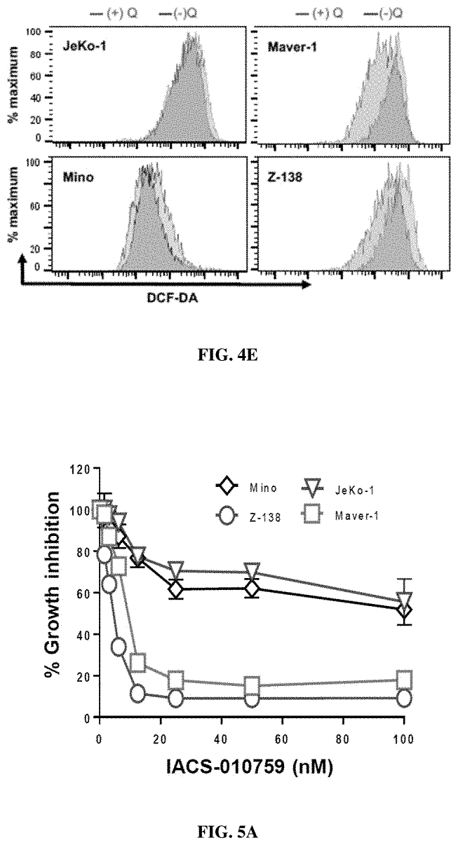

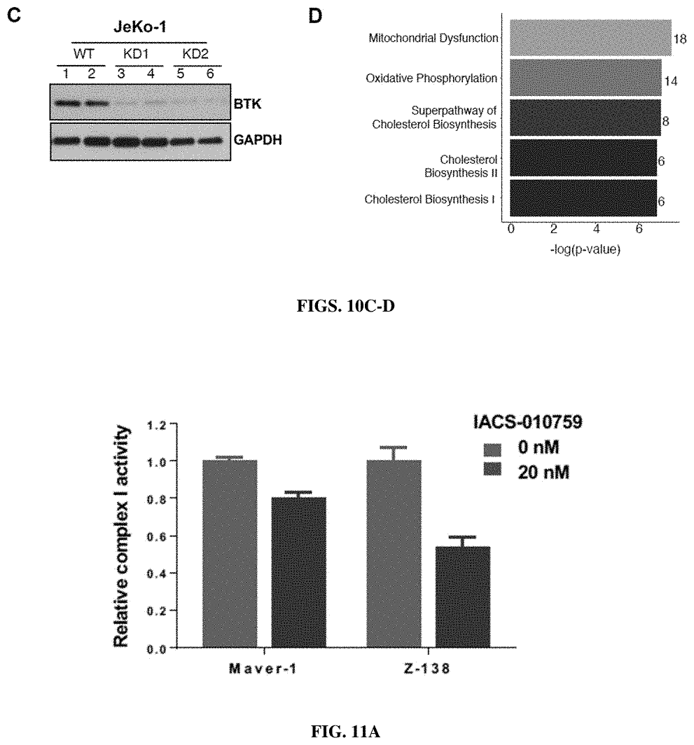

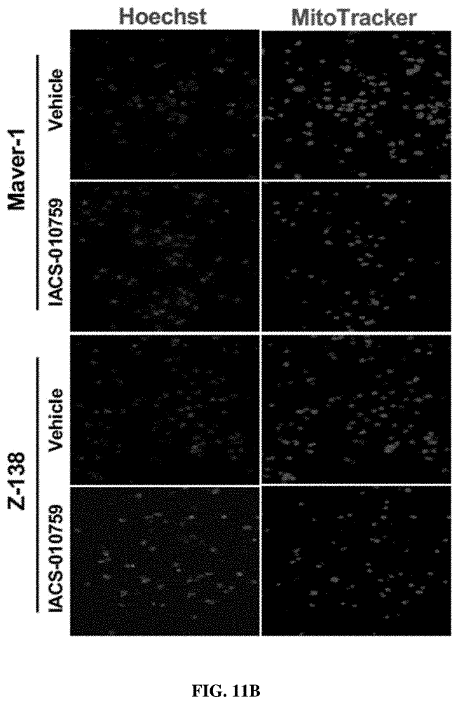

In support of the hypothesis that ibrutinib-resistant MCL cells rely on glutamine-fueled OXPHOS, ibrutinib-resistant MCL cell lines displayed higher basal (FIG. 4A) and ATP-coupled (FIG. 4B) oxygen consumption rates (OCR), an indicator of OXPHOS activity, in comparison to ibrutinib-sensitive MCL cell lines. Targeted metabolomics analysis showed significantly higher levels of the metabolite α-KG in the resistant cells (fold change: 2.9698; two-sample t-test; p=0.0196; FIG. 4C), suggesting enhanced glutamine metabolism. Incorporation of α-KG into the TCA cycle is a major anaplerotic step in proliferating cells and is critical for the maintenance of TCA cycle function (Lukey et al., 2013). To corroborate this observation, glutaminase (GLS), the enzyme that converts glutamine to glutamate, a precursor of α-KG, was overexpressed in ibrutinib-resistant MCL (fold change: 1.2094; two-sample t-test; p<0.0001; FIG. 4D). Glutamine uptake was also upregulated in the ibrutinib-resistant MCL cell lines (FIG. 10A), and glutamine deprivation (FIG. 4E) or blockade of glutamine metabolism with aminooxyacetate (AOA) (FIG. 10B), a glutaminolysis inhibitor, resulted in marked induction of reactive oxygen species (ROS) and energy stress compared with the sensitive cell lines. Lastly, ibrutinib resistance was conferred on the ibrutinib-sensitive JeKo-1 cell line utilizing CRISPR-Cas 9 to alter BTK, the target of ibrutinib (Li et al., under review). In the two genetically manipulated clones, deletions were introduced in one BTK allele, leaving the other allele intact. Because BTK is X-linked and the JeKo-1 cells were derived from a female patient, the inactivated X-chromosome most likely remained unaffected by the gRNA and remained wild type, with very little BTK protein production from this X-inactivated allele, creating a BTK knockdown model (FIG. 10C) that is resistant to ibrutinib. Proteomics analysis of the JeKo-BTK KD clones compared with the JeKo-1 parental line showed increased expression of OXPHOS-associated proteins (FIG. 10D), suggesting that ibrutinib resistance is associated with increased OXPHOS expression. These results demonstrate that upregulated mitochondrial OXPHOS energy production may contribute to ibrutinib resistance in MCL.

### Example 4—Targeting the OXPHOS Pathway Overcomes Ibrutinib Resistance in MCL

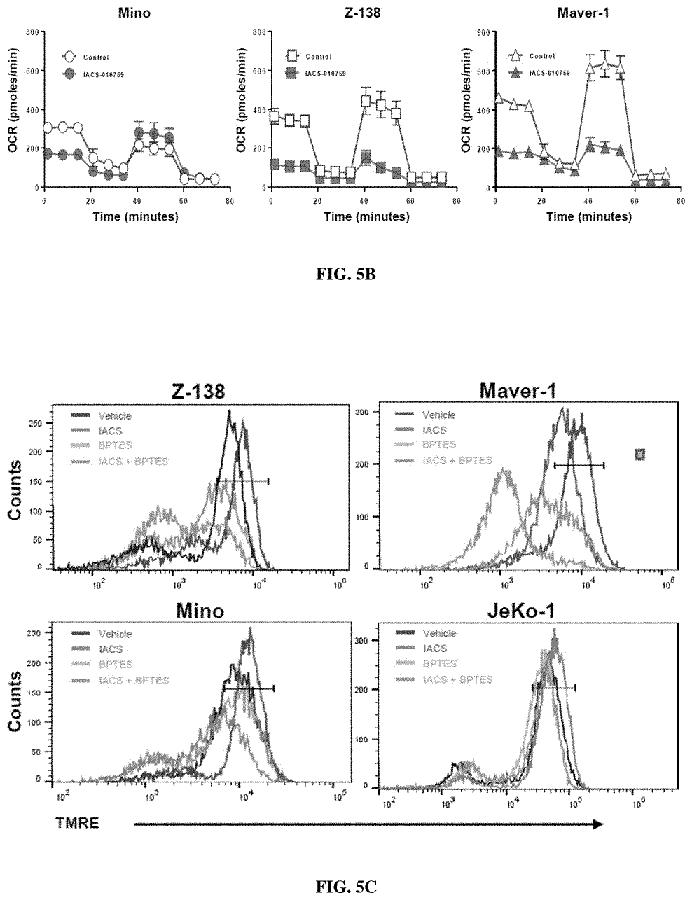

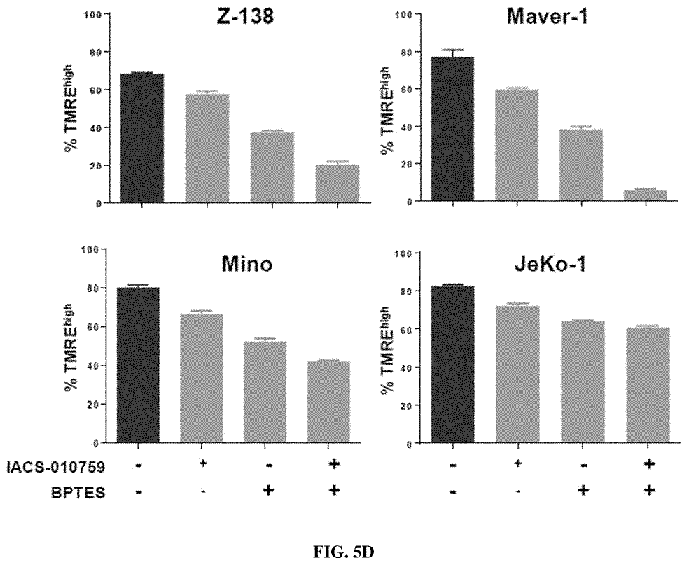

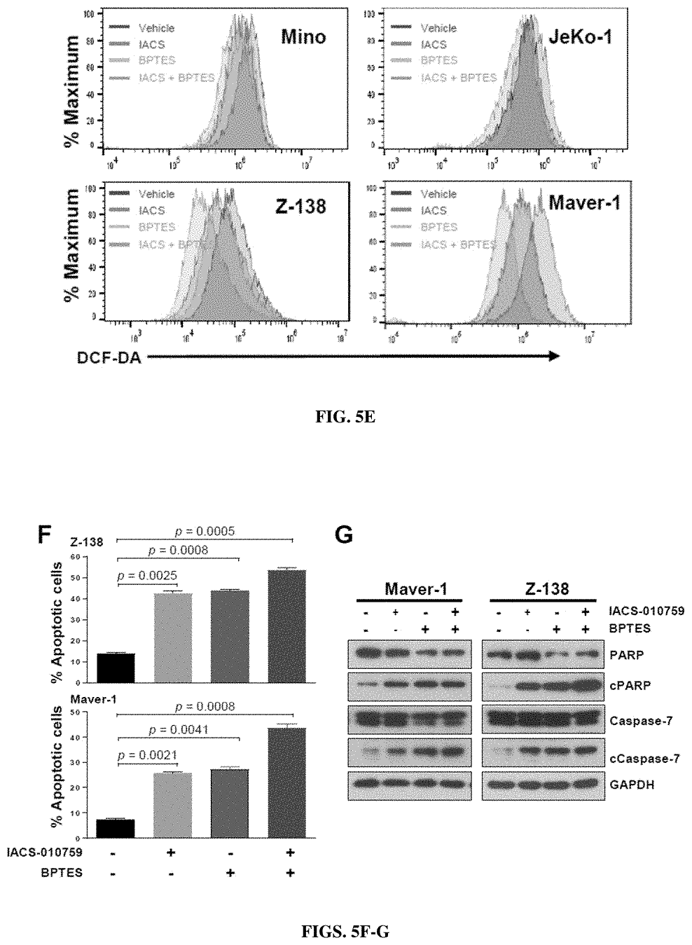

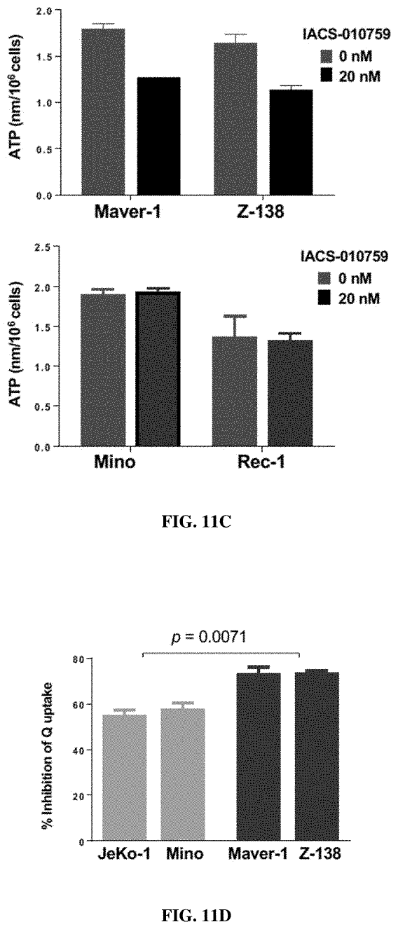

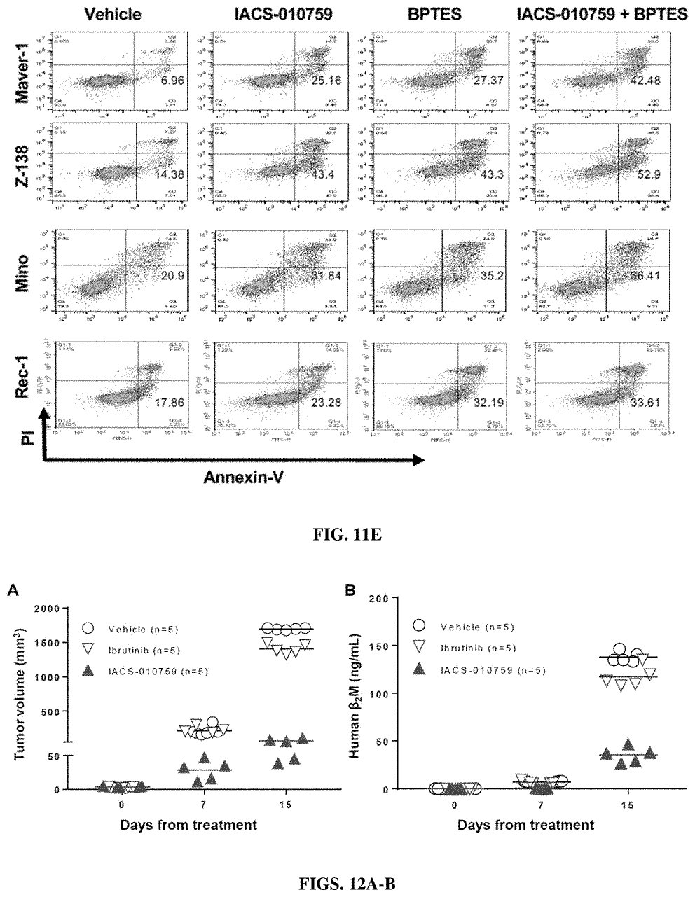

Targeting the PI3K/AKT/mTOR pathway in relapsed/refractory lymphoma is currently being actively investigated in multiple clinical trials but has resulted in only moderate clinical success thus far. Moreover, C-MYC is considered “undruggable” (Dang et al., 2017; Posternak & Cole, 2016). Based on the evidence showing that the TCA/OXPHOS pathway appears to be a prominent energy metabolism pathway in ibrutinib-resistant MCL cells, the anti-MCL effects of suppressing the OXPHOS pathway in refractory MCL cells was assessed. OXPHOS is a critical mitochondrial process that generates ATP and intermediates to meet the requirements for cell growth. During oxidative phosphorylation, electrons are transferred from electron donors to acceptors through the ETC in redox reactions that release energy to form ATP. Therefore, a novel inhibitor of ETC complex I, IACS-010759, developed by MD Anderson Cancer Center (Molina et al., 2018) that is currently in Phase I clinical trials for acute myeloid leukemia (NCT02882321) and solid tumors and lymphoma (NCT03291938) was used. Intriguingly, IACS-010759 inhibited the proliferation of the ibrutinib-resistant MCL cell lines (Z-138 and Maver-1) in a dose-dependent manner at nanomolar concentrations but had very little effect on the ibrutinib-sensitive MCL cell lines (FIG. 5A), with an approximately 10-fold difference in IC₅₀ values between the resistant and sensitive cell lines. Confirming the activity of IACS-010759, ETC complex I activity was reduced in ibrutinib-resistant MCL cell lines after treatment with the inhibitor (FIGS. 11A & 11B). In addition, treatment with the OXPHOS inhibitor reduced the OCR in the ibrutinib-resistant MCL cell lines, Z-138 and Maver-1, but had little effect on the OCR of the ibrutinib-sensitive MCL cell line, Mino, compared with the DMSO-treated control (FIG. 5B).

To probe the mechanisms by which IACS-010759 inhibited proliferation of the ibrutinib-resistant MCL cells, the effects of the complex I inhibitor on mitochondrial potential and ATP production were examined. IACS-010759 treatment decreased both mitochondrial potential (FIGS. 5C & 5D) and ATP production (FIG. 11C). Moreover, IACS-010759 exposure resulted in greater glutamine uptake inhibition in the ibrutinib-resistant MCL cells compared with the sensitive cells through a currently unknown mechanism (FIG. 11D). Based on our RPPA profiling and metabolomics of ibrutinib-sensitive versus-resistant MCL cell lines showing increased GLS and α-KG levels (FIGS. 4C & 4D), it was hypothesized that the MCL cells may still rely on glutamine anaplerosis for energy production and survival. Glutamine-derived a-KG is an important precursor for other cellular energy pathways, such as nucleotide biosynthesis and lipid synthesis, that promote cell survival and growth. As expected, inhibition of glutamine metabolism with the allosteric GLS1-selective inhibitor bis-2-(5-phenylacetamido-1,3,4-thiadiazol-2-yl)ethyl sulfide (BPTES) further reduced the mitochondrial potential in combination with IACS-010759 (FIGS. 5C & 5D). Inhibition of mitochondrial ETC complex I has been suggested to result in growth inhibition by positively modulating ROS (Chen et al., 2007). To determine whether ROS production is altered by IACS-010759, ROS levels post-IACS-010759 treatment (25 nM) were measured in Maver-1 and Z-138 MCL cell lines. As predicted, IACS-010759 induced ROS production in the ibrutinib-resistant cell lines but not in the -sensitive cell lines (FIG. 5E), suggesting that IACS-010759 disrupts the critical redox balance that is maintained by NADH in ibrutinib-resistant MCL. Further suppression of glutamine metabolism with the addition of BPTES resulted in even greater ROS production in the ibrutinib-resistant MCL cell lines Maver-1 and Z-138, likely attributable to decreased production of glutathione.

Increases in ROS production are associated with apoptosis induction (Chen et al., 2007; Higuchi et al., 1998); therefore, the apoptotic effects of IACS-010759 were examined in the ibrutinib-sensitive and -resistant MCL cell lines to determine if apoptosis underlies the growth inhibition observed with IACS-010759 treatment. As shown in FIGS. 5F & 11E, single agent IACS-010759 treatment of 72 hours resulted 23% and 42% apoptosis in the ibrutinib-resistant Maver-1 and Z-138 MCL cell lines, respectively. IACS-010759 single agent treatment induced caspase 7 activation and PARP cleavage (FIG. 5G). The observed apoptosis levels were not sufficient to explain the drastic growth inhibition observed with IACS-010759 treatment, suggesting that additional mechanisms, such as upregulated glutaminolysis, continue to sustain survival. Single agent treatment with BPTES in ibrutinib-resistant MCL cell lines yielded similar levels of apoptosis relative to single agent IACS-010759 (25% and 44% versus 23% and 42% in the ibrutinib-resistant Maver-1 and Z-138 MCL cells, respectively) (FIGS. 5F & 11E) and also induced caspase 7 activation and PARP cleavage (FIG. 5G). Indeed, co-administration of BPTES to inhibit glutamine metabolism induced caspase 7 activation and PARP cleavage (FIG. 5G) and resulted in significantly increased apoptosis in the ibrutinib-resistant MCL cell lines (41% in Maver-1 cells and 53% in Z-138 cells), again suggesting that ibrutinib-resistant MCL cells produce sufficient reducing equivalents to quench ROS; however, combinatory treatment with IACS-010759 and BPTES disrupts this critical balance.

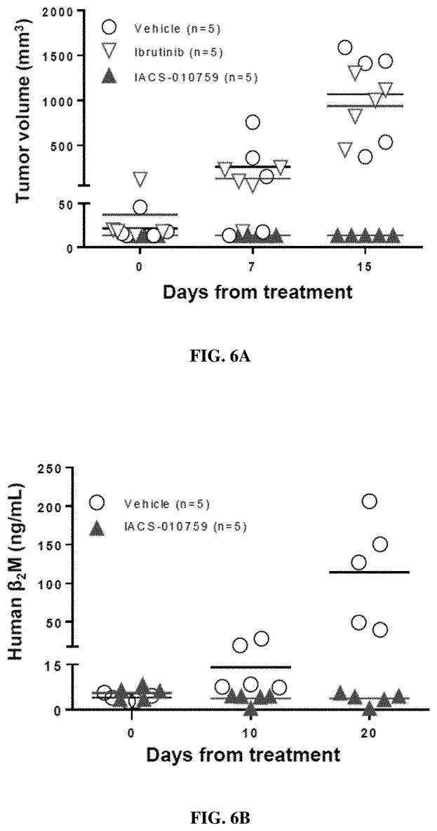

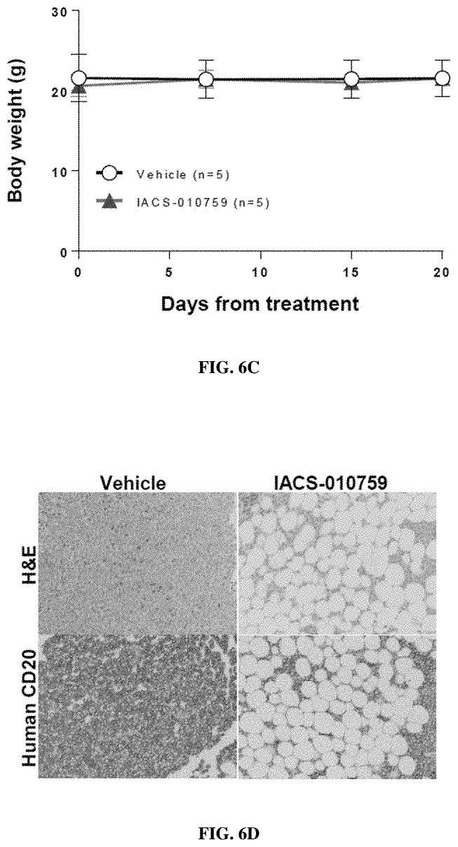

To confirm the observed in vitro anti-MCL effects of IACS-010759, its effects on tumor growth and proliferation were examined in an ibrutinib-resistant MCL PDX mouse model (FIG. 6A). Single agent IACS-010759 treatment at 10 mg/kg oral gavage for 5 consecutive days/week completely prevented tumor growth compared with the vehicle control as shown by measuring tumor volume (FIG. 6A, n=5, p<0.0001) and human β₂M levels (FIG. 6B, n=5; p<0.0001) throughout treatment. No apparent toxicities were observed in the IACS-010759-treated MCL PDX mice. For example, body weight was not significantly different between vehicle control and IACS-010759 treatment group (FIG. 6C; p=0.3304). H&E and anti-CD20 staining of dissected tissue post-treatment suggested repopulation of the tumor area with fatty deposits; however, a small number of CD20+ cells remained after treatment (FIG. 6D).

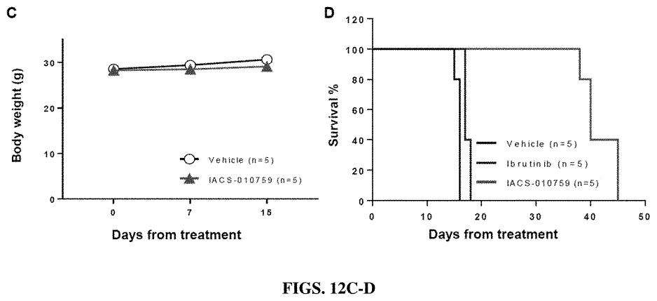

To confirm the therapeutic effects of IACS-010759 in vivo, another ibrutinib-resistant B-cell lymphoma PDX model was developed using a clinically refractory/relapsed high-grade B-cell lymphoma patient sample. Single agent IACS-010759 but not ibrutinib significantly inhibited tumor growth as demonstrated by the measurement of tumor volume (FIG. 12A, ibrutinib vs vehicle, p=0.7227, IACS-010759 vs vehicle, p<0.0001) and human (32M levels (FIG. 12B, ibrutinib vs vehicle, p=0.6911, IACS-010759 vs vehicle, p=0.0003). Body weight was also not significantly different between the vehicle and IACS-010759 treatment groups (FIG. 12C; p=0.1964). Additionally, IACS-010759 significantly prolonged the survival of the ibrutinib-resistant PDX mice (FIG. 12D; n=5, IACS-010759 vs vehicle, p=0.0035, IACS-010759 vs ibrutinib, p=0.0035) compared with the vehicle- and ibrutinib-treated mice, with IACS-010759 conferring a survival benefit of greater than 20 days.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Adzhubei et al., “Predicting functional effect of human missense mutations using PolyPhen-2,” Curr. Protoc. Hum. Genet., Chapter 7, Unit 7.20 (2013).
Altman et al., “From Krebs to clinic: glutamine metabolism to cancer therapy,” Nat. Rev. Cancer, 16:749 (2016).
Baltazar et al., “Monocarboxylate transporters as targets and mediators in cancer therapy response,” Histol. Histopathol., 29:1511-1524 (2014).
Benjamini & Yekutieli, “The control of the false discovery rate in multiple testing under dependency,” Ann. Stat., 29:1165-1188 (2001).
Cardoso et al., “70-Gene signature as an aid to treatment decisions in early-stage breast cancer,” New Engl. J. Med., 375:717-729, 2016.
Caro et al., “Metabolic signatures uncover distinct targets in molecular subsets of diffuse large B cell lymphoma,” Cancer Cell, 22:547-560 (2012).
Carter et al., “Identifying Mendelian disease genes with the variant effect scoring tool,” BMC Genomics., 14(Suppl 3):S3 (2013).
Cheah et al., “Patients with mantle cell lymphoma failing ibrutinib are unlikely to respond to salvage chemotherapy and have poor outcomes,” Ann. Oncol., 26:1175-1179 (2015).
Chen et al., “Mitochondrial electron-transport-chain inhibitors of complexes I and II induce autophagic cell death mediated by reactive oxygen species,” J. Cell. Sci., 120:4155-4166 (2007).
Chiron et al., “Cell-cycle reprogramming for PI3K inhibition overrides a relapse-specific C481S BTK mutation revealed by longitudinal functional genomics in mantle cell lymphoma,” Cancer Discovery, 4:1022-1035 (2014).
Choi et al., “Predicting the functional effect of amino acid substitutions and indels,” PLoS One, 7:e46688 (2012).
Chun & Fay, “Identification of deleterious mutations within three human genomes,” Genome Res., 19:1553-1561 (2009).
Cibulskis et al., “Sensitive detection of somatic point mutations in impure and heterogeneous cancer samples,” Nat. Biotechnol., 31:213-219 (2013).
Clot et al., “A gene signature that distinguishes convention and leukemic non-nodal mantle cell lymphoma helps predict outcome,” Blood, 132:413-422, 2018.
Csibi et al., “The mTORC1/S6K1 pathway regulates glutamine metabolism through the eIF4B-dependent control of c-Myc translation,” Curr. Biol., 24:2274-2280 (2014).
Dang, “MYC, metabolism, cell growth, and tumorigenesis,” Cold Spring Harb. Perspect. Med., 3:a014217 (2013).
Dang et al., “Drugging the ‘undruggable’ cancer targets,” Nat. Rev. Cancer, 17:502-508 (2017).
Davydov et al., “Identifying a high fraction of the human genome to be under selective constraint using GERP++,” PLoS Comput. Biol., 6:e1001025 (2010).
DeBerardinis et al., “Beyond aerobic glycolysis: transformed cells can engage in glutamine metabolism that exceeds the requirement for protein and nucleotide synthesis,” Proc. Natl. Acad. Sci. U.S.A., 104:19345-19350 (2007).
DeLuca et al., “RNA-SeQC: RNA-seq metrics for quality control and process optimization,” Bioinformatics, 28:1530-1532 (2012).
DePristo et al., “A framework for variation discovery and genotyping using next-generation DNA sequencing data,” Nat. Genet., 43:491-498 (2011).
Dobin et al., “STAR: ultrafast universal RNA-seq aligner,” Bioinformatics, 29:15-21 (2013).
Dong et al., “Comparison and integration of deleteriousness prediction methods for nonsynonymous SNVs in whole exome sequencing studies,” Hum. Mol. Genet., 24:2125-2137 (2015).
Fernandez et al., “Pathogenesis of mantle-cell lymphoma: all oncogenic roads lead to dysregulation of cell cycle and DNA damage response pathways,” J. Clin. Oncol., 23:6364-6369 (2005).
Galimberti & Petrini, “Temsirolimus in the treatment of relapsed and/or refractory mantle cell lymphoma,” Cancer Manag. Res., 2:181-189 (2010).
Hess et al., “Phase III study to evaluate temsirolimus compared with investigator's choice therapy for the treatment of relapsed or refractory mantle cell lymphoma,” J. Clin. Oncol., 27:3822-3829 (2009).
Higuchi et al., “Regulation of reactive oxygen species-induced apoptosis and necrosis by caspase 3-like proteases,” Oncogene, 17:2753-2760 (1998).
Jain et al., “Long-term outcomes and mutation profiling of patients with mantle cell lymphoma (MCL) who discontinued ibrutinib,” Br. J. Haematol., 183:578-587, 2018.
Kahl et al., “A phase 1 study of the PI3Kdelta inhibitor idelalisib in patients with relapsed/refractory mantle cell lymphoma (MCL),” Blood, 123:3398-3405 (2014).
Kircher et al., “A general framework for estimating the relative pathogenicity of human genetic variants,” Nat. Genet., 46:310-315 (2014).
Kumar et al., “Predicting the effects of coding non-synonymous variants on protein function using the SIFT algorithm,” Nat. Protoc., 4:1073-1081 (2009).
Kuppers, “Mechanisms of B-cell lymphoma pathogenesis,” Nat. Rev. Cancer, 5:251-262 (2005).
Lampa et al., “Glutaminase is essential for the growth of triple-negative breast cancer cells with a deregulated glutamine metabolism pathway and its suppression synergizes with mTOR inhibition,” PLoS One, 12:e0185092 (2017).
Lannutti et al., “CAL-101, a p110delta selective phosphatidylinositol-3-kinase inhibitor for the treatment of B-cell malignancies, inhibits PI3K signaling and cellular viability,” Blood, 117:591-594 (2011).
Li & Durbin, “Fast and accurate short read alignment with Burrows-Wheeler transform,” Bioinformatics, 25:1754-1760 (2009).
Liberzon et al., “The Molecular Signatures Database (MSigDB) hallmark gene set collection,” Cell. Syst., 1:417-425 (2015).
Liu et al., “dbNSFP v3.0: A One-Stop Database of Functional Predictions and Annotations for Human Nonsynonymous and Splice-Site SNVs,” Hum. Mutat., 37:235-241 (2016).
Lorenzi et al., “A curated census of autophagy-modulating proteins and small molecules: candidate targets for cancer therapy,” Autophagy, 10:1316-1326 (2014).
Lukey et al., “Therapeutic strategies impacting cancer cell glutamine metabolism,” Future Med. Chem., 5:1685-1700 (2013).
Matre et al., “Inhibiting glutaminase in acute myeloid leukemia: metabolic dependency of selected AML subtypes,” Oncotarget, 7:79722-79735 (2016).
Molina et al., “An inhibitor of oxidative phosphorylation exploits cancer vulnerability,” Nat. Med., 24:1036-1046, 2018.
Myklebust et al., “Distinct patterns of B-cell receptor signaling in non-Hodgkin lymphomas identified by single-cell profiling,” Blood, 129:759-770 (2017).
Nicklin et al., “Bidirectional transport of amino acids regulates mTOR and autophagy,” Cell, 136:521-534 (2009).
Olshen et al., “Circular binary segmentation for the analysis of array-based DNA copy number data,” Biostatistics, 5:557-572 (2004).
Perez-Galan et al., “Mantle cell lymphoma: biology, pathogenesis, and the molecular basis of treatment in the genomic era,” Blood, 117:26-38 (2011).
Pinheiro et al., “Role of monocarboxylate transporters in human cancers: state of the art,” J. Bioenerg. Biomembr., 44:127-139 (2012).
Posternak & Cole, “Strategically targeting MYC in cancer,” F1000Res., 5:408 (2016).
Pusapati et al., “mTORC1-Dependent Metabolic Reprogramming Underlies Escape from Glycolysis Addiction in Cancer Cells,” Cancer Cell, 29:548-562 (2016).
Quang et al., “DANN: a deep learning approach for annotating the pathogenicity of genetic variants,” Bioinformatics, 31:761-763 (2015).
Rahal et al., “Pharmacological and genomic profiling identifies NF-kappaB-targeted treatment strategies for mantle cell lymphoma,” Nat. Med., 20:87-92 (2014).
Reva et al., “Predicting the functional impact of protein mutations: application to cancer genomics,” Nucleic Acids Res., 39:e118 (2011).
Rimmer et al., “Integrating mapping-, assembly- and haplotype-based approaches for calling variants in clinical sequencing applications,” Nat. Genet., 46:912-918 (2014).
Saba et al., “Pathogenic role of B-cell receptor signaling and canonical NF-kappaB activation in mantle cell lymphoma,” Blood, 128:82-92 (2016).
Schwarz et al., “MutationTaster2: mutation prediction for the deep-sequencing age,” Nat. Methods, 11:361-362 (2014).
Shihab et al., “An integrative approach to predicting the functional effects of non-coding and coding sequence variation,” Bioinformatics, 31:1536-1543 (2015).
Subramanian et al., “Gene set enrichment analysis: a knowledge-based approach for interpreting genome-wide expression profiles,” Proc. Natl. Acad. Sci. U.S.A., 102:15545-15550 (2005).
Tabe et al., “Class IA PI3K inhibition inhibits cell growth and proliferation in mantle cell lymphoma,” Acta Haematol., 131:59-69 (2014).
Tarrado-Castellarnau et al., “Oncogenic regulation of tumor metabolic reprogramming,” Oncotarget, 7:62726-62753 (2016).
Thorvaldsdottir et al., “Integrative Genomics Viewer (IGV): high-performance genomics data visualization and exploration,” Brief Bioinform., 14:178-192 (2013).
Vander Heiden et al., “Understanding the Warburg effect: the metabolic requirements of cell proliferation,” Science, 324:1029-1033 (2009).
van Geldermalsen et al., “ASCT2/SLC1A5 controls glutamine uptake and tumour growth in triple-negative basal-like breast cancer,” Oncogene, 35:3201-3208 (2016).
Wang et al., “A severe combined immunodeficient-hu in vivo mouse model of human primary mantle cell lymphoma,” Clin. Cancer Res., 14:2154-2160 (2008).
Wang et al., “Targeting BTK with ibrutinib in relapsed or refractory mantle-cell lymphoma,” N. Engl. J. Med., 369:507-516 (2013).
Wisniewski et al., “High recovery FASP applied to the proteomic analysis of microdissected formalin fixed paraffin embedded cancer tissues retrieves known colon cancer markers,” J. Proteome Res., 10:3040-3049 (2011).
Witzig et al., “Phase II trial of single-agent temsirolimus (CCI-779) for relapsed mantle cell lymphoma,” J. Clin. Oncol., 23:5347-5356 (2005).
Woyach, “Patterns of resistance to B cell-receptor pathway antagonists in chronic lymphocytic leukemia and strategies for management,” Hematology Am. Soc. Hematol. Educ. Program, 2015:355-360 (2015).
Woyach et al., “BTK(C481S)-Mediated Resistance to Ibrutinib in Chronic Lymphocytic Leukemia,” J. Clin. Oncol., 35:1437-1443 (2017).
Ye et al., “Pindel: a pattern growth approach to detect break points of large deletions and medium sized insertions from paired-end short reads,” Bioinformatics, 25:2865-2871 (2009).
Yee et al., “Phase I/II study of the mammalian target of rapamycin inhibitor everolimus (RAD001) in patients with relapsed or refractory hematologic malignancies,” Clin. Cancer Res., 12:5165-5173 (2006).
Zhang et al., “Intratumor heterogeneity in localized lung adenocarcinomas delineated by multiregion sequencing,” Science, 346:256-259 (2014).
Zhang et al., “B-Cell Lymphoma Patient-Derived Xenograft Models Enable Drug Discovery and Are a Platform for Personalized Therapy,” Clin. Cancer Res., 23:4212-4223 (2017).
Zhang et al., “Metabolic reprogramming toward oxidative phosphorylation identifies a therapeutic target for mantle cell lymphoma,” Sci. Transl. Med., 11:eaau1167, 2019.
Zhao et al., “Unification of de novo and acquired ibrutinib resistance in mantle cell lymphoma.,” Nat. Commun., 8:14920 (2017).

What is claimed is:

1. A method of treating a patient having a lymphoma, the method comprising:
(a) detecting whether the patient's cancer has an ibrutinib-sensitive or ibrutinib-resistant gene expression profile relative to a reference sample by:
(i) obtaining or having obtained a sample of the patient's lymphoma;
(ii) performing or having performed an assay on the sample to measure an expression level of a plurality of genes in the sample, wherein each gene in the plurality of genes is selected from Table 1; and
(iii) generating an expression profile based on a comparison between the expression level of the plurality of genes in the sample and a corresponding expression level obtained from a reference sample;
(b) selecting or having selected a treatment for the patient based on whether the patient's cancer has an ibrutinib-sensitive or ibrutinib-resistant gene expression profile; and
(c) administering or having administered to the patient a therapeutically effective amount of:
(i) ibrutinib, if the patient is determined to have an ibrutinib-sensitive lymphoma; or
(ii) an inhibitor of oxidative phosphorylation, a BH3-mimetic, a noncovalent BTK inhibitor, or a CAR-T therapy, if the patient is determined to have an ibrutinib-resistant lymphoma.

2. The method of claim 1, wherein the patient is classified as having an ibrutinib-resistant lymphoma if the expression of SLC16A1 is upregulated.

3. The method of claim 1, wherein the patient is classified as having an ibrutinib-resistant lymphoma if:
(a) the expression of at least two of the following genes is upregulated: HR, HMBS, HN1, PYCR1, SLC26A8, SEPT3, INPP5J, SLC1A5, CCDC86, CTPS1, TOMM40, TFRC, TRIP13, LRP8, SQLE, HIVEP3, FADS1, TTLL12, SLC25A19, RCC1, NPM3, CCT5, DDX21, MTHFD2, SLC16A1, and NME1; and/or
(b) the expression of at least two of the following genes is downregulated: LCN8, CD84, OR2C1, CD8A, COL4A4, COL4A3, MFHAS1, SSH3, MYO10, PTPRN2, SPATA18, PCSK1, BAZ2B, PSO3, NEB, PLCXD2, ZNF433, ADAMTS10, ARMCX4, TVP23C, ACMSD, AGRP, IQSEC3, RNGTT, FAAH2, CCDC173, SCIMP, PRAG1, TOR4A, ZNF395, RBPMS, BFSP2, LDLRAD4, A4FALT, KBTBD6, FAM159A, and ARSD.

4. The method of claim 1, wherein the patient is classified as having an ibrutinib-sensitive lymphoma if:
(a) the expression of at least two of the following genes is downregulated: HR, HMBS, HN1, PYCR1, SLC26A8, SEPT3, INPP5J, SLC1A5, CCDC86, CTPS1, TOMM40, TFRC, TRIP13, LRP8, SQLE, HIVEP3, FADS1, TTLL12, SLC25A19, RCC1, NPM3, CCT5, DDX21, MTHFD2, SLC16A1, and NME1; and/or
(b) the expression of at least two of the following genes is upregulated: LCN8, CD84, OR2C1, CD8A, COL4A4, COL4A3, MFHAS1, SSH3, MYO10, PTPRN2, SPATA18, PCSK1, BAZ2B, PSO3, NEB, PLCXD2, ZNF433, ADAMTS10, ARMCX4, TVP23C, ACMSD, AGRP, IQSEC3, RNGTT, FAAH2, CCDC173, SCIMP, PRAG1, TOR4A, ZNF395, RBPMS, BFSP2, LDLRAD4, A4FALT, KBTBD6, FAM159A, and ARSD.

5. The method of claim 1, wherein the expression level of the plurality of genes is measured by detecting a level of mRNA transcribed from the plurality of genes.

6. The method of claim 5, wherein the mRNA level is detected by microarray, RT-PCR, qRT-PCR, nanostring assay, or in situ hybridization.

7. The method of claim 1, wherein the expression level of the plurality of genes is measured by detecting a level of cDNA produced from reverse transcription of mRNA transcribed from the plurality of genes.

8. The method of claim 1, wherein the expression level of the plurality of genes is measured by detecting a level of polypeptide encoded by the plurality of genes.

9. The method of claim 1, wherein the sample is a formalin-fixed, paraffin-embedded sample.

10. The method of claim 1, wherein the sample is a fresh frozen sample.

11. The method of claim 1, wherein the reference sample is a sample from a healthy subject.

12. The method of claim 1, wherein the reference sample is a sample of non-cancerous cells obtained from the patient.

13. The method of claim 1, wherein the inhibitor of oxidative phosphorylation is IACS-010759.

14. The method of claim 1, wherein the BH3-mimetic is venetoclax.

15. The method of claim 1, wherein the noncovalent BTK inhibitor is Loxo-305.

16. The method of claim 1, wherein the CAR-T therapy targets CD19.

17. The method of claim 1, wherein if the patient is determined to have an ibrutinib-resistant lymphoma, then the method further comprising administering a therapeutically effective amount of a glutaminase inhibitor to the patient.

18. The method of claim 1, wherein if the patient is determined to have an ibrutinib-resistant lymphoma, then the method further comprising administering a therapeutically effective amount of a mTOR inhibitor to the patient.

19. The method of claim 1, wherein the lymphoma is mantle cell lymphoma.

20. A composition comprising a set of nanostring probes that hybridize to the target sequence for at least 40, 45, 50, 55, or 60 of the genes listed in Table 1.
